(19) United States
(12) Patent Application Publication
(10) Pub. No.: US 2022/0401292 A1
(43) Pub. Date: Dec. 22, 2022

(54) LEG MASSAGER

(71) Applicant: DAITO ELECTRIC MACHINE INDUSTRY COMPANY LIMITED, Higashi-Osaka-shi, Osaka (JP)

(72) Inventors: Tetsuya ISHIKAWA, Higashi-Osaka-shi (JP); Shinsaku SHIMIZU, Higashi-Osaka-shi (JP)

(73) Assignee: DAITO ELECTRIC MACHINE INDUSTRY COMPANY LIMITED, Higashi-Osaka-shi, Osaka (JP)

(21) Appl. No.: 17/771,995

(22) PCT Filed: Jul. 20, 2020
(86) PCT No.: PCT/JP2020/028034

§ 371 Date: Apr. 26, 2022

(30) Foreign Application Priority Data
Dec. 11, 2019 (JP) 2019-223451

(51) Int. Cl. A61H 15/00; A61H 15/02; A61H 39/04; A61H 7/00

(52) U.S. Cl. CPC A61H 15/0078; A61H 15/02; A61H 39/04; A61H 7/007; A61H 2201/164; A61H 2015/0014; A61H 2201/5064

(57) ABSTRACT

A leg massager achieves easy changing of massaging positions and is also adaptable to user's legs of varying lengths by using a position-adjustable massaging system for performing massage on massage target areas of a user's legs. The leg massager includes a lower massaging system for massaging a massage target area of the user's leg including at least the user's foot; an upper massaging system for massaging an upper massage target area located above the massage target area which is massaged by the lower massaging system; a rockably supporting system that supports the upper massaging system for rocking motion about a horizontal-pointing axis in a front-rear direction; and a vertically moving system that permits up-and-down movement of the upper massaging system.

### TECHNICAL FIELD

The present invention relates to a leg massager for applying a pressing force for massage to user's leg, in particular, a calf-thigh range of the leg.

### BACKGROUND ART

There is a heretofore known leg massager incorporating a massaging system for performing massage on massage target areas of user's leg in the range from a toe-to-heel region, i.e. the foot, to the calf located between the ankle and the knee (refer to Patent Literature 1, for instance).

### PRIOR ART REFERENCE

### Citation List

Patent Literature 1: Japanese Unexamined Patent Publication JP-A 2011-103988

### SUMMARY OF INVENTION

### Technical Problem

The variety of user's demands for massaging equipment has increased in recent years. For example, there are user requests for a massager incorporating a system that allows a user to have massage on wide areas of his or her leg in the range from, in addition to the foot, the lower side of the calf, through the entire calf, to the thigh, while sitting in a chair, etc.

Furthermore, users of varying body shapes have different leg lengths. For example, a short person of small build has a relatively short leg length, whereas a tall person of large build has a relatively long leg length. This fact has led to user demands for a massager incorporating a system capable of adapting to user's legs of varying lengths.

The present invention has been devised in view of the circumstances as discussed supra, and accordingly its object is to provide a leg massager that achieves easy changing of massaging positions and is also capable of adapting to user's legs of varying lengths by using a position-adjustable massaging system for performing massage on massage target areas of user's legs.

### Solution to Problem

In order to accomplish the described object, the following technical means is adopted for the present invention to be carried into effect.

A leg massager pursuant to the present invention comprises: a lower massaging system for massaging a massage target area of user's leg including at least user's foot; an upper massaging system for massaging an upper massage target area located above the massage target area which is massaged by the lower massaging system; a rockably supporting system that supports the upper massaging system for rocking motion about a horizontal-pointing axis in a front-rear direction; and a vertically moving system that permits up-and-down movement of the upper massaging system.

In the present invention, it is preferable that the vertically moving system includes a mechanism that brings the upper massaging system to a stop in a predetermined position during the time the vertically moving system is moving the upper massaging system upward.

In the present invention, it is preferable that the rockably supporting system is situated above the vertically moving system, and rockably supports a base-end side of a first massaging member disposed in the upper massaging system so that a front-end side of the first massaging member can be raised rearward via a horizontal-pointing rock shaft.

In the present invention, it is preferable that the first massaging member is folded forward toward a second massaging member disposed in the lower massaging system via the rockably supporting system, at which time the first massaging member is in a position with its front-end side directed forward.

In the present invention, it is preferable that the first massaging member is raised, with its front-end side moved away from the second massaging member disposed in the lower massaging system, via the rockably supporting system, at which time the first massaging member is in an obliquely upward and forward-facing position.

In the present invention, it is preferable that the first massaging member is raised rearward, with its front-end side moved away from the second massaging member disposed in the lower massaging system, via the rockably supporting system, at which time the first massaging member is in an obliquely upward and rearward-facing position.

In the present invention, it is preferable that the first massaging member is folded forward toward the second massaging member disposed in the lower massaging system via the rockably supporting system, and the first massaging member in the forward-leaning position is then moved upward via the vertically moving system, at which time the first massaging member is in a position with its front-end side directed forward.

In the present invention, it is preferable that the first massaging member is moved upward via the vertically moving system, and the first massaging member in the up position is then rocked upward via the rockably supporting system, at which time the first massaging member is in an obliquely upward and forward-facing position.

In the present invention, it is preferable that the first massaging member is moved upward via the vertically moving system, and the first massaging member in the up position is then rocked upward and rearward via the rockably supporting system, at which time the first massaging member is in an obliquely upward and rearward-facing position.

In the present invention, it is preferable that the rockably supporting system is configured to rock the first massaging member in a manner permitting successive changing of its position from the original forward-leaning position to the obliquely upward and forward-facing position, and from there to the obliquely upward and rearward-facing position.

In the present invention, it is preferable to provide a stop mechanism that stops the vertically moving system upon the first massaging member reaching a predetermined position during its upward movement.

In the present invention, it is preferable that the stop mechanism comprises: an upper detection section for detecting an arrival of the upwardly-moving first massaging member at a predetermined position; and a stop section that stops the vertically moving system in accordance with a result of detection by the upper detection section.

In the present invention, it is preferable to provide a safety mechanism that stops the vertically moving system and/or the rockably supporting system in case of trouble during a downward movement of the first massaging member.

In the present invention, it is preferable that the safety mechanism comprises: a lower detection section for detecting a hindrance to a downward movement of the first massaging member; and a stop section that stops the vertically moving system and/or the rockably supporting system in accordance with a result of detection by the lower detection section.

In the present invention, it is preferable that the first massaging member is allowed to move upward, move downward, and rotate in a condition where user's foot is held between the second massaging members.

In the present invention, it is preferable that the first massaging member is shaped in an elongated plate, and that the first massaging member is provided at its mid-portion with a rotatable pressing member, which is free to rotate about an axis pointing in a direction longitudinally of the first massaging member, for producing rotational pressure to perform pressing massage on user's leg as a massage target.

### Advantageous Effects of Invention

The present invention provides a leg massager that achieves easy changing of massaging positions and is also capable of adapting to user's legs of varying lengths by using a position-adjustable massaging system for performing massage on massage target areas of user's legs.

### BRIEF DESCRIPTION OF DRAWINGS

FIG. 1 is a side view of the leg massager pursuant to the present invention, illustrating vertical movement and back-and-forth rocking motion of the upper massaging system.

FIG. 2 is a side view showing a position with the lowered massaging section of the upper massaging system substantially horizontally oriented (first position).

FIG. 3 is a side view showing a position with the lowered massaging section of the upper massaging system facing obliquely upward and forward (second position).

FIG. 4 is a side view showing a position with the lowered massaging section of the upper massaging system inclined slightly rearward relative to the vertical (third position).

FIG. 5 is a side view showing a position with the lifted massaging section of the upper massaging system substantially horizontally oriented (fourth position).

FIG. 6 is a side view showing a position with the lifted massaging section of the upper massaging system facing obliquely upward and forward (fifth position).

FIG. 7 is a side view showing a position with the lifted massaging section of the upper massaging system inclined slightly rearward relative to the vertical (sixth position).

FIG. 8 is a front perspective view showing the leg massager pursuant to the present invention assuming the position with the lowered massaging section of the upper massaging system substantially horizontally oriented (first position).

FIG. 9 is a front perspective view showing the leg massager pursuant to the present invention assuming the position with the lifted massaging section of the upper massaging system inclined slightly rearward relative to the vertical (sixth position).

FIG. 10 is a front perspective view showing the internal structures of the leg massager pursuant to the present invention assuming the position with the lifted massaging section of the upper massaging system inclined slightly rearward relative to the vertical (sixth position).

FIG. 11 is a rear perspective view showing the internal structures of the leg massager pursuant to the present invention assuming the position with the lowered massaging section of the upper massaging system substantially horizontally oriented (first position).

FIG. 12 is a view showing a rotatable pressing member-equipped first massaging member of the leg massager.

FIG. 13 is a plate showing the penetrating rotatable pressing member exposed from the first massaging member.

### DESCRIPTION OF EMBODIMENTS

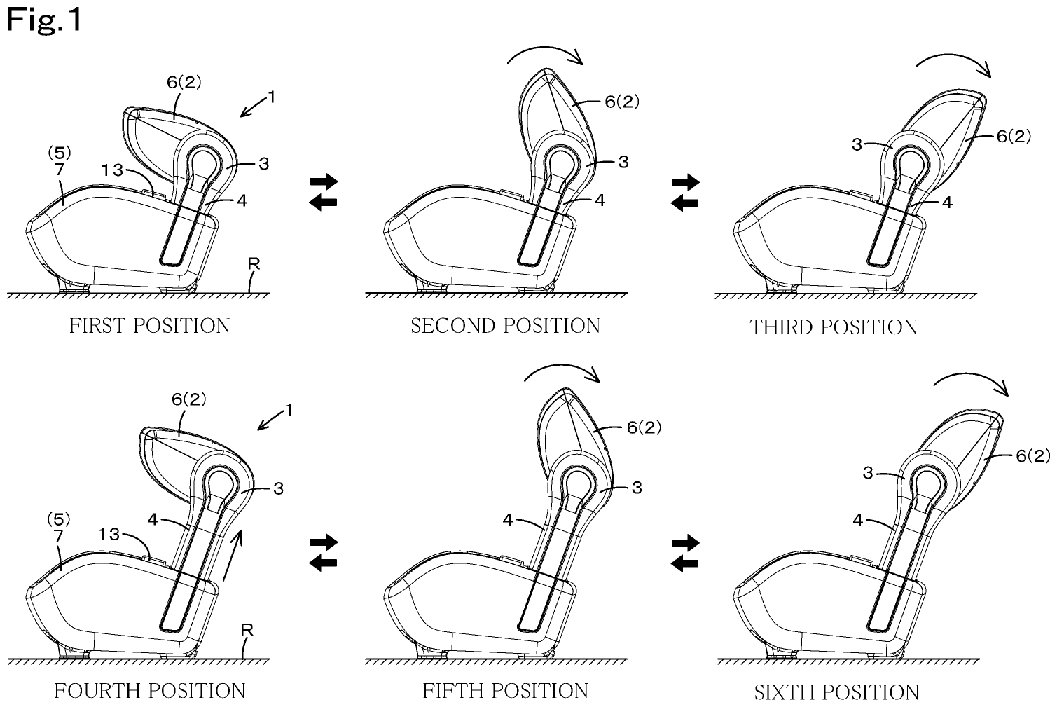

An embodiment of a leg massager 1 pursuant to the present invention will now be described with reference to FIGS. 1 to 11. The following embodiment is given by way of example of carrying the present invention into effect, and therefore not intended to be limiting of the structural features of the present invention. Moreover, some constituent components are omitted from FIGS. 1 to 11 for clarity of illustration.

The leg massager 1 according to this embodiment effects changing and adjustment of a massaging position to perform massage on various areas (massage target areas) of user's leg L.

In this embodiment, the leg L refers to users' leg areas in the range from thigh T, through calf C, to foot F. The “thigh T” refers to that part of human leg L located above the knee, the “calf C” refers to that part of the leg L located below the knee and located above the ankle, and the “foot F” refers to that part of the leg L located below the ankle and extending from the heel to the toe.

Moreover, as seen from some drawings for reference, terms of orientation such as forward and rearward (front-rear direction), horizontal (right-left direction), and upward and downward (vertical direction) are defined in accordance with the view of a user sitting in a chair, etc. with his/her legs L placed in the leg massager 1. In the following description, the calf C may also be called “first massage target area”, and the thigh T may also be called “second massage target area”.

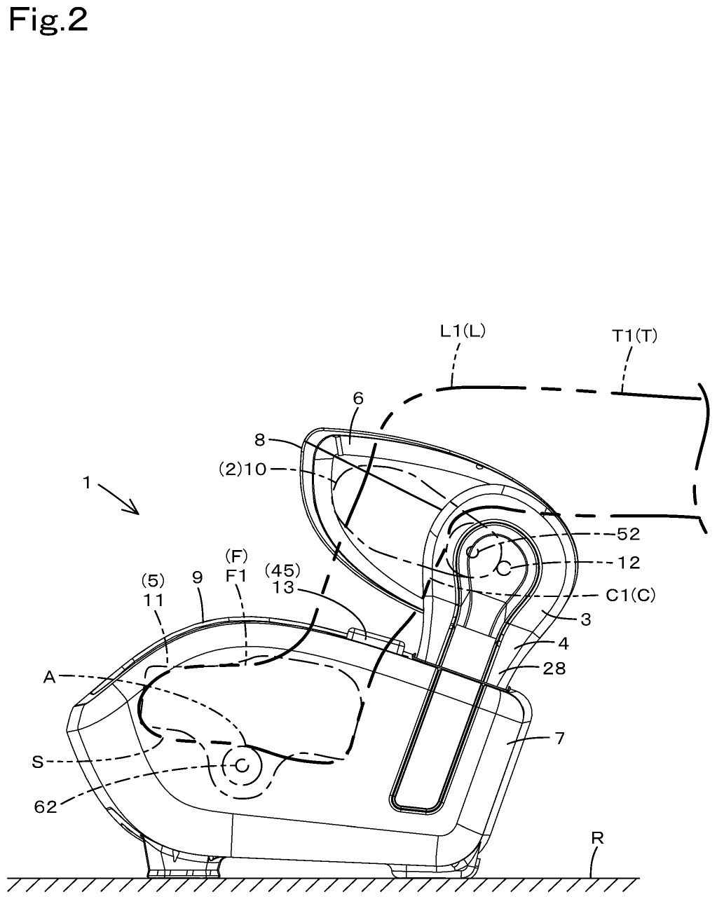

For example, the upper half of FIG. 1, and FIGS. 2 to 4 are explanatory drawings illustrating upper massaging positions for cases where a person having a relatively short leg length uses the leg massager 1 pursuant to the present invention.

A position as shown in the left-hand drawing in the upper half of FIG. 1 and also in FIG. 2 with a first massaging member 10, which is disposed inside an upper casing 6, substantially horizontally oriented corresponds to an upper massaging position defined as “first position” intended for calf C1 of a user whose leg L1 is relatively short.

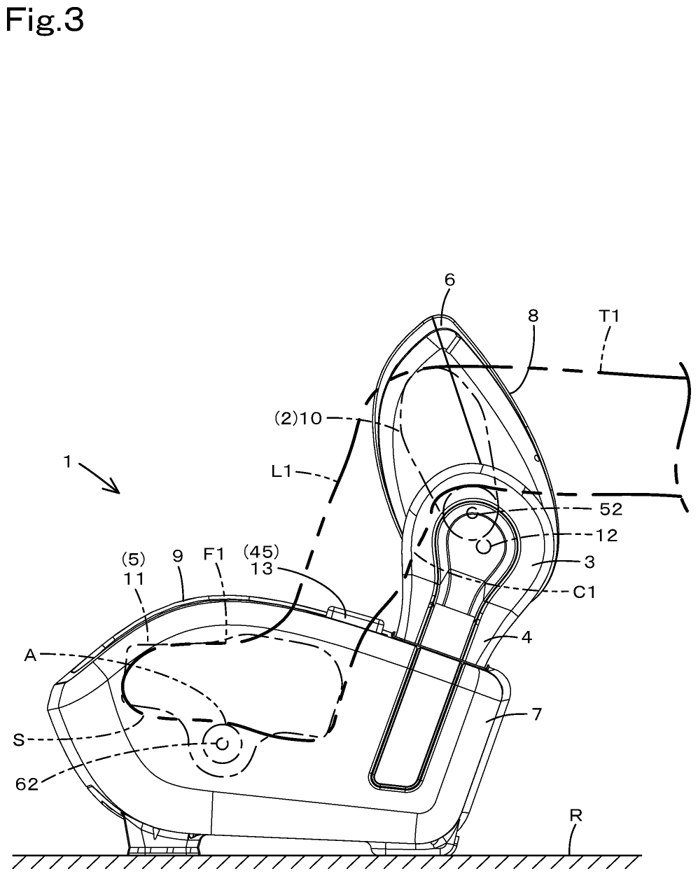

A position as shown in the middle drawing in the upper half of FIG. 1 and also in FIG. 3 with the first massaging member 10 inside the upper casing 6 directed forward and obliquely upward corresponds to an upper massaging position defined as “second position” intended for the upper part of the calf C1, as well as the knee, of a user whose leg L1 is relatively short.

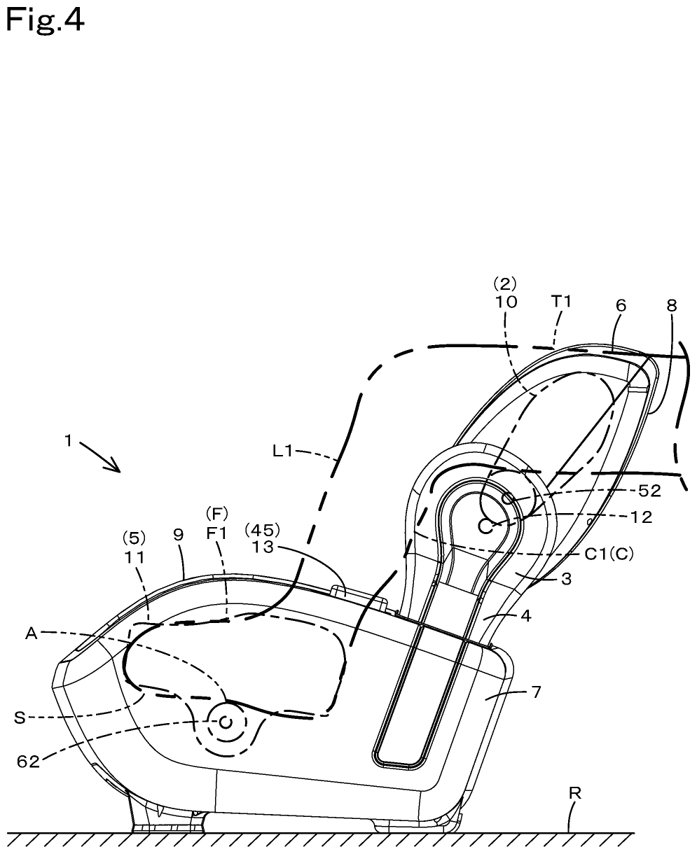

A position as shown in the right-hand drawing in the upper half of FIG. 1 and also in FIG. 4 with the first massaging member 10 inside the upper casing 6 inclined slightly rearward relative to the vertical corresponds to an upper massaging position defined as “third position” intended for thigh T1 of a user whose leg L1 is relatively short.

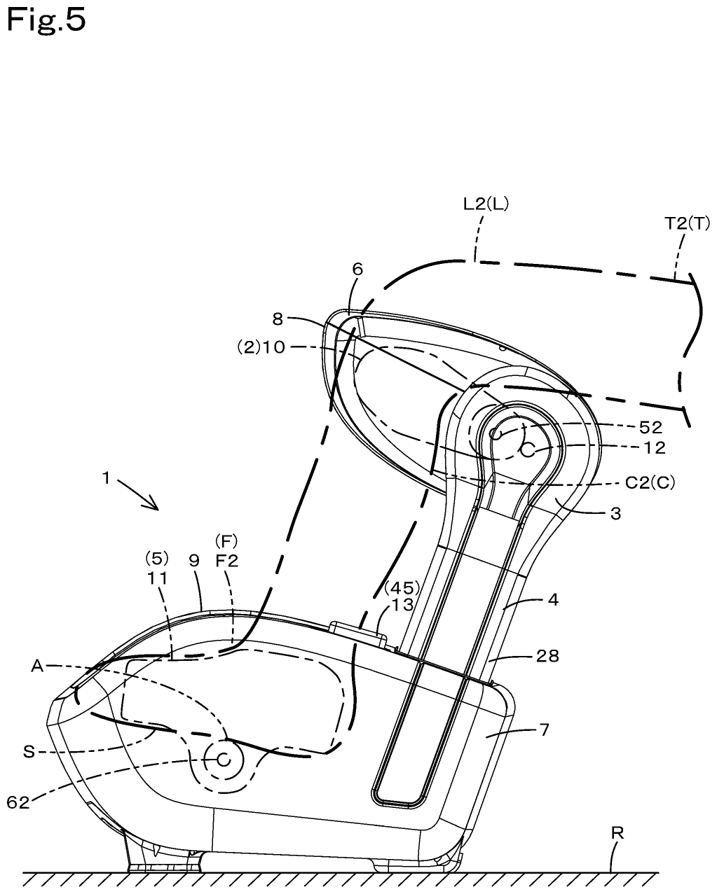

Moreover, for example, the lower half of FIG. 1, and FIGS. 5 to 7 are explanatory drawings illustrating upper massaging positions for cases where a person having a relatively long leg length uses the leg massager 1 pursuant to the present invention.

A position as shown in the left-hand drawing in the lower half of FIG. 1 and also in FIG. 5 with the first massaging member 10 inside the upper casing 6 substantially horizontally oriented corresponds to an upper massaging position defined as “fourth position” intended for calf C2 of a user whose leg L2 is relatively long.

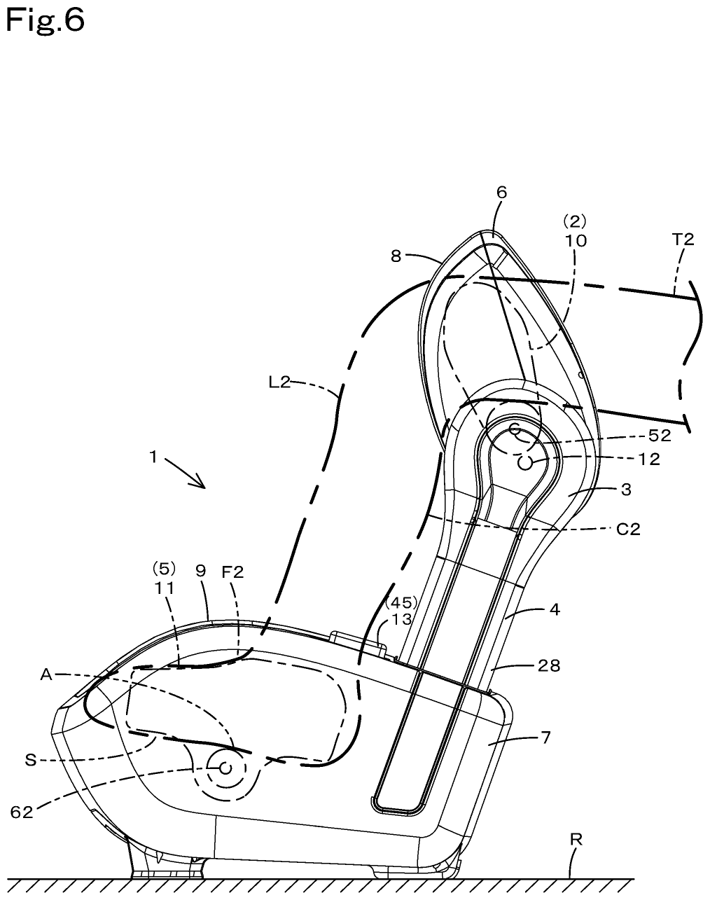

A position as shown in the middle drawing in the lower half of FIG. 1 and also in FIG. 6 with the first massaging member 10 inside the upper casing 6 directed forward and obliquely upward corresponds to an upper massaging position defined as “fifth position” intended for the upper part of the calf C2, as well as the knee, of a user whose leg L2 is relatively long.

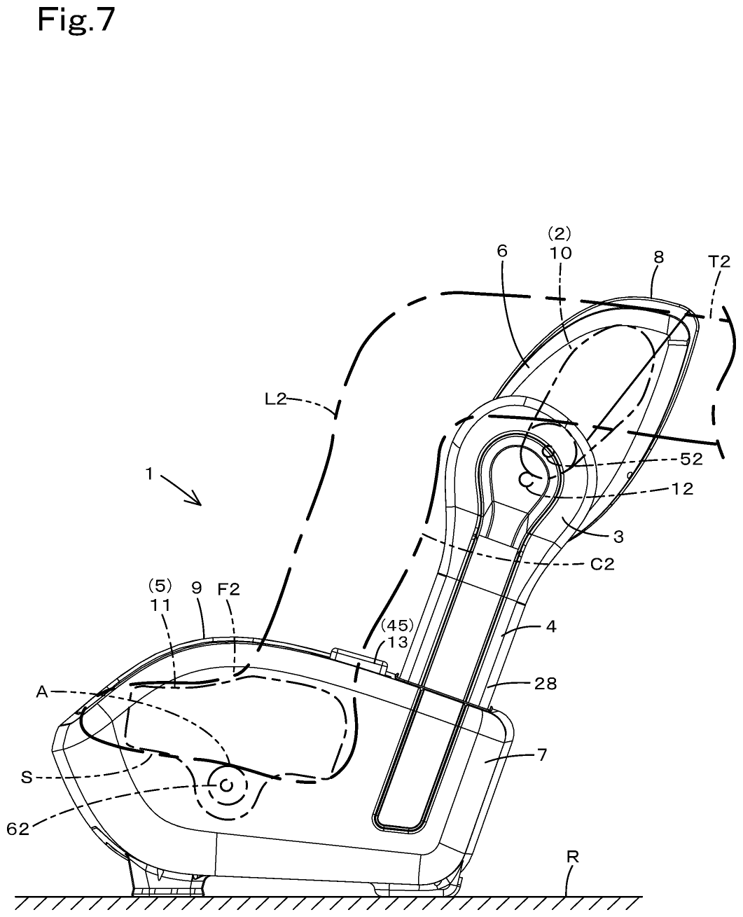

A position as shown in the right-hand drawing in the lower half of FIG. 1 and also in FIG. 7 with the first massaging member 10 inside the upper casing 6 inclined slightly rearward relative to the vertical corresponds to an upper massaging position defined as “sixth position” intended for thigh T2 of a user whose leg L2 is relatively long.

Referring first to FIGS. 1 to 7, the operation of an upper massaging system 2 constituting the leg massager 1 pursuant to the present invention, and more specifically the “first to sixth positions”, will be described in detail. The description as to the structure of the leg massager 1 pursuant to the present invention will be given later.

The leg massager 1 pursuant to the present invention comprises: the upper massaging system 2 for performing massage on massage target areas in the range from the calf C through the thigh T; a rockably supporting system 3 that supports the upper massaging system 2 for rocking motion about a horizontal-pointing axis in the front-rear direction; a vertically moving system 4 that permits up-and-down movement of the upper massaging system 2; and a lower massaging system 5 for performing massage on massage target areas of user's leg L including at least the foot F.

The upper massaging system 2 is housed in the upper casing 6, and, the lower massaging system 5 is housed in a lower casing 7.

Referring first to the upper half of FIG. 1 and also FIGS. 2 to 4, the conditions of the upper casing 6 corresponding to the “first position”, the “second position”, and the “third position” will be described. For example, the “first to third positions” are intended for the leg L1 of a user of relatively small build.

For example, in attaining the “first position” shown in the left-hand drawing in the upper half of FIG. 1 and also in FIG. 2, the upper casing 6 is folded forward toward the lower casing 7 by the rockably supporting system 3. That is, the upper casing 6 has its main body oriented substantially horizontally, and its front-end side directed forward.

The upper casing 6 is formed with first massaging recesses 8. At the “first position”, each first massaging recess 8 has an open top, an open front, and an open bottom. Moreover, the lower casing 7 is formed with second massaging recesses 9, each in a condition of having an open bottom.

The user places his or her legs L1 into the massager so that each calf C1 is received in the first massaging recess 8 of the upper casing 6, and each foot F1 is received in the second massaging recess 9 of the lower casing 7.

More specifically, as shown in FIG. 2, the calf C1 is sandwiched between the first massaging members 10 of the upper massaging system 2. At the “first position”, the first massaging members 10 apply a lateral squeezing force for pressing massage to the calf C1 (first massage target area) placed in the first massaging recess 8.

The size of user's calf C1 is determinative of the pressing position of the first massaging member 10 in a longitudinal direction. Moreover, the upper massaging system 2 is secured by the rockably supporting system 3.

Meanwhile, the foot F1 is sandwiched between the second massaging members 11 of the lower massaging system 5. At the “first position”, the second massaging members 11 apply a lateral squeezing force for pressing massage to the foot F1 (second massage target area), i.e. the toe-to-heel region of user's leg, placed in the second massaging recess 9.

A shift from this position to the “second position” is effected by rocking the upper casing 6 upward about the axis of a rock shaft 12 of the upper massaging system 2.

In attaining the “second position” shown in the middle drawing in the upper half of FIG. 1 and also in FIG. 3, the upper casing 6 is raised with its front-end side directed forward and upward in a direction away from the lower casing 7 by the rockably supporting system 3. That is, the upper casing 6 is slightly lifted into an obliquely upward and forward-facing position. At the “second position”, the first massaging recess 8 has an open top, an open front, and an open rear.

More specifically, as shown in FIG. 3, a knee-side part of the thigh T1 is sandwiched between the first massaging members 10 of the upper massaging system 2. At the “second position”, the first massaging members 10 apply a lateral squeezing force for pressing massage to the knee-side part of the thigh T1 placed in the first massaging recess 8. The “second position” shown in FIG. 3 may be considered to be a position of transition from the “first position” to the “third position”.

The size of user's thigh T1 is determinative of the pressing position of the first massaging member 10 in the longitudinal direction. Moreover, the upper massaging system 2 is secured by the rockably supporting system 3.

A shift from this position to the “third position” is effected by rocking the upper casing 6 further upward about the axis of the rock shaft 12 of the upper massaging system 2.

In attaining the “third position” shown in the right-hand drawing in the upper half of FIG. 1 and also in FIG. 4, the upper casing 6 is raised with its front-end side directed rearward and upward in a direction away from the lower casing 7 by the rockably supporting system 3. That is, the upper casing 6 is tilted back into an obliquely upward and rearward-facing position. At the “third position”, the first massaging recess 8 has an open top, an open front, and an open rear.

More specifically, as shown in FIG. 4, a leg joint-side part of the thigh T1 is sandwiched between the first massaging members 10 of the upper massaging system 2. At the “third position”, the first massaging members 10 apply a lateral squeezing force for pressing massage to the leg joint-side part of the thigh T1 placed in the first massaging recess 8.

The size of user's thigh T1 is determinative of the pressing position of the first massaging member 10 in the longitudinal direction. Moreover, the upper massaging system 2 is secured by the rockably supporting system 3.

Thus, the upper massaging system 2 can be moved to the desired position simply by raising the upper casing 6, ensuring easy changing of the upper massaging position.

The upper massaging position can be changed (returned) from the “third position” to the “first position” or the “second position” by reversing the described procedure.

The upper casing 6 (upper massaging system 2) can be rocked in the front-rear direction and locked in the desired position, e.g. any of the “first to third positions”, ensuring changing of the upper massaging position.

That is, for example, at the “first position” shown in the left-hand drawing in the upper half of FIG. 1 and also in FIG. 2, the right-hand and left-hand first massaging recesses 8 receive the right and left calves C1, respectively.

At the “second position” shown in the middle drawing in the upper half of FIG. 1 and also in FIG. 3, the right-hand and left-hand first massaging recesses 8 receive the right and left thighs T1 (knee-side part), respectively.

At the “third position” shown in the right-hand drawing in the upper half of FIG. 1 and also in FIG. 4, the right-hand and left-hand first massaging recesses 8 receive the right and left thighs T1 (leg joint-side part), respectively. In this way, the massager performs massage in different massaging positions, namely the “first to third positions”, for example. That is, the leg massager 1 pursuant to the present invention is suited for the leg L1 of a user of relatively small build (having a relatively short leg length).

The upper massaging positions such as the “first to third positions” as employed in this embodiment are one example and therefore not intended to be limiting of the angularity of the upper massaging system 2 as exemplified herein.

Moreover, use can be made of an arrangement that enables the upper casing 6 to rock in the front-rear direction for back-and-forth travel between the “first position” and the “third position”. This makes it possible to massage the calf C1 area and the thigh T1 area in succession.

That is, preferably, the rockably supporting system 3 is configured to rock the upper casing 6 rearward in a manner permitting successive changing of its position from the original forward-leaning position to the obliquely upward and forward-facing position away from the lower casing 7, and from there to the obliquely upward and rearward-facing position.

Referring next to the lower half of FIG. 1 and also FIGS. 5 to 7, the conditions of the upper casing 6 corresponding to the “fourth position”, the “fifth position”, and the “sixth position” will be described. For example, the “fourth to sixth positions” are intended for the leg L2 of a user of relatively large build.

For example, in attaining the “fourth position” shown in the left-hand drawing in the lower half of FIG. 1 and also in FIG. 5, the upper casing 6, while being folded forward by the rockably supporting system 3, is moved upward by the vertically moving system 4.

That is, the upper casing 6 is spaced a predetermined height above the lower casing 7, with its main body oriented substantially horizontally and its front-end side directed forward. At the “fourth position”, the first massaging recesses 8 of the upper casing 6 each have an open top, an open front, and an open bottom.

The user places his or her legs L2 into the massager so that each calf C2 is received in the first massaging recess 8 of the upper casing 6, and each foot F2 is received in the second massaging recess 9 of the lower casing 7.

More specifically, as shown in FIG. 5, the calf C2 is sandwiched between the first massaging members 10 of the upper massaging system 2. At the “fourth position”, the first massaging members 10 apply a lateral squeezing force for pressing massage to the calf C2 (first massage target area) placed in the first massaging recess 8.

The size of user's calf C2 is determinative of the pressing position of the first massaging member 10 in the longitudinal direction. Moreover, the upper massaging system 2 is secured by the rockably supporting system 3.

Meanwhile, the foot F2 is sandwiched between the second massaging members 11 of the lower massaging system 5. At the “fourth position”, the second massaging members 11 apply a lateral squeezing force for pressing massage to the foot F1 (second massage target area), i.e. the toe-to-heel region of user's leg, placed in the second massaging recess 9. With user's foot held between the second massaging members 11, even when the first massaging member 10 undergoes a shift from the “first position” to the “fourth position”, there is effectively avoided inconvenience such as an accidental lift of user's leg. The same holds true for cases where the first massaging member 10 undergoes a shift to the “fifth position” or the “six position” as will hereafter be described. Also, with user's foot held between the second massaging members 11, even when the first massaging member undergoes a shift to the “second position” or the “third position” as will hereafter be described, there is effectively avoided inconvenience such as an accidental lift of user's leg.

Preferably, a pressure point-massaging element is so located as to face the sole of user's foot. This makes it possible to, on the shift of the first massaging member 10 from the “fourth position” to the “first position”, perform more efficacious pressure-point massage on the sole of the foot being held between the second massaging members 11. For example, it is very desirable to provide means for enabling the pressure point-massaging element to retract downward in sync with the shift of the first massaging member from the “fourth position” to the “first position” when pressure point-massaging pressure becomes excessive.

A shift from the “fourth position” to the “fifth position” is effected by rocking the upper casing 6 upward about the axis of the rock shaft 12 of the upper massaging system 2.

In attaining the “fifth position” shown in the middle drawing in the lower half of FIG. 1 and also in FIG. 6, after being moved upward by the vertically moving system 4, the upper casing 6 is raised with its front-end side directed forward and upward by the rockably supporting system 3.

That is, the upper casing 6, while being spaced a predetermined height above the lower casing 7, is slightly lifted into an obliquely upward and forward-facing position. At the “fifth position”, the first massaging recess 8 has an open top, an open front, and an open rear.

More specifically, as shown in FIG. 6, a knee-side part of the thigh T2 is sandwiched between the first massaging members 10 of the upper massaging system 2. At the “fifth position”, the first massaging members 10 apply a lateral squeezing force for pressing massage to the knee-side part of the thigh T2 placed in the first massaging recess 8. The “fifth position” shown in FIG. 6 may be considered to be a position of transition from the “fourth position” to the “sixth position”.

The size of user's thigh T2 is determinative of the pressing position of the first massaging member 10 in the longitudinal direction. Moreover, the upper massaging system 2 is secured by the rockably supporting system 3.

A shift from this position to the “sixth position” is effected by rocking the upper casing 6 further upward about the axis of the rock shaft 12 of the upper massaging system 2.

In attaining the “sixth position” shown in the right-hand drawing in the lower half of FIG. 1 and also in FIG. 7, after being moved upward by the vertically moving system 4, the upper casing 6 is raised with its front-end side directed rearward and upward by the rockably supporting system 3.

That is, the upper casing 6, while being spaced a predetermined height above the lower casing 7, is tilted back into an obliquely upward and rearward-facing position. At the “sixth position”, the first massaging recess 8 has an open top, an open front, and an open rear.

More specifically, as shown in FIG. 7, a leg joint-side part of the thigh T2 is sandwiched between the first massaging members 10 of the upper massaging system 2. At the “sixth position”, the first massaging members 10 apply a lateral squeezing force for pressing massage to the leg joint-side part of the thigh T2 placed in the first massaging recess 8.

The size of user's thigh T2 is determinative of the pressing position of the first massaging member 10 in the longitudinal direction. Moreover, the upper massaging system 2 is secured by the rockably supporting system 3.

Thus, the upper massaging system 2 can be moved to the desired position simply by raising the upper casing 6 which has been moved to a higher position spaced a predetermined height above the lower casing 7, ensuring easy changing of the upper massaging position.

The upper massaging position can be changed (returned) from the “sixth position” to the “fourth position” or the “fifth position” by reversing the described procedure.

After being moved upward, the upper casing 6 (upper massaging system 2) can be rocked in the front-rear direction and locked in the desired position, e.g. any of the “fourth to sixth positions”, thereby accomplishing changing of the upper massaging position.

That is, for example, at the “fourth position” shown in the left-hand drawing in the lower half of FIG. 1 and also in FIG. 5, the right-hand and left-hand first massaging recesses 8 receive the right and left calves C2, respectively.

At the “fifth position” shown in the middle drawing in the lower half of FIG. 1 and also in FIG. 6, the right-hand and left-hand first massaging recesses 8 receive the right and left thighs T2 (knee-side part), respectively.

At the “sixth position” shown in the right-hand drawing in the lower half of FIG. 1 and also in FIG. 7, the right-hand and left-hand first massaging recesses 8 receive the right and left thighs T2 (leg joint-side part), respectively. In this way, the massager performs massage in different massaging positions, namely the “fourth to sixth positions”, for example. That is, the leg massager 1 pursuant to the present invention is also suited for the leg L2 of a user of relatively large build (having a relatively long leg length).

The upper massaging positions such as the “fourth to sixth positions” as employed in this embodiment are one example and therefore not intended to be limiting of the angularity of the upper massaging system 2 as exemplified herein.

Moreover, use can be made of an arrangement that enables the upper casing 6 to move upward and then rock in the front-rear direction for back-and-forth travel between the “fourth position” and the “sixth position”. This makes it possible to massage the calf C2 area and the thigh T2 area in succession.

That is, preferably, after the upper casing 6 in its forward-leaning position toward the lower casing 7 is moved upward by using the vertically moving system 4, the rockably supporting system 3 rocks the upper casing 6 rearward in a manner permitting successive changing of its position from the original forward-leaning position to the obliquely upward and forward-facing position, and from there to the obliquely upward and rearward-facing position.

For the rising (rocking) movement of the upper casing 6, for example, the rockably supporting system 3 may be provided with a mechanism that enables the upper casing 6 (upper massaging system 2) to “rise” and “lean forward” automatically (which will hereafter be described in detail).

That is, preferably, the rockably supporting system 3 has a mechanism that enables the upper casing 6 to “rise” and “lean forward” automatically, so that the upper casing 6 (upper massaging system 2) can be automatically rocked in the front-rear direction for back-and-forth movement between the calf C area and the thigh T area via the rockably supporting system 3. This makes it possible to massage, in addition to the foot F, leg areas in the range from the calf C through the thigh T.

The foot F placed in the second massaging recess 9 of the lower casing 7 may be accidentally lifted in response to the automatic back-and-forth rocking motion of the upper casing 6 (upper massaging system 2) in the front-rear direction. It is therefore preferable to provide an arrangement that enables the paired second massaging members 11 to press and hold the foot F1 firmly. This eliminates the occurrence of a lift of the foot F.

Moreover, for the upward and downward movement of the upper casing 6, the vertically moving system 4 may be provided with a mechanism that enables the upper casing 6 to move up and down automatically (which will hereafter be described in detail). This enables the massager to easily adapt to user's legs of varying lengths and thus massage the leg L properly regardless of its length (the length of a below-knee part of the leg L, in particular), whether it be short or long.

It is also preferable to provide an arrangement that enables the automatic “rising” and “forward-leaning” movement of the upper casing 6 and the “upward” and “downward” movement thereof to be effected in concert with each other.

The following describes the structure of the leg massager 1 according to this embodiment.

The leg massager 1, which is placed on a floor R, etc., serves to massage both of the right and left legs L, as massage target areas, of a user sitting in a chair, etc. The leg massager 1 is designed to perform simultaneous kneading and squeezing massage on different areas of the leg L, namely the foot F and any of the calf C and the thigh T, for example.

Moreover, the leg massager 1 has an arrangement for producing vibrating and pressing motion to perform finger-pressure massage on the sole S and the arch (of the foot) A of a user. The leg massager 1 also has an arrangement capable of performing thermomassage on the leg L.

The leg massager 1 comprises: the lower massaging system 5 for performing massage on massage target areas of user's leg L including at least the foot F; the upper massaging system 2 for performing massage on massage target areas (calf C, thigh T, etc.) located above the massage target areas to be massaged by the lower massaging system 5; the rockably supporting system 3 that supports the upper massaging system 2 for rocking motion about a horizontal-pointing axis in the front-rear direction; and the vertically moving system 4 that permits up-and-down movement of the upper massaging system 2.

The leg massager 1 further comprises: a safety mechanism 13 that stops the vertically moving system 4 in case of trouble during the downward movement of the upper casing 6; and a stop mechanism 14 that stops the vertically moving system 4 upon the upper casing 6 reaching a predetermined position during its upward movement.

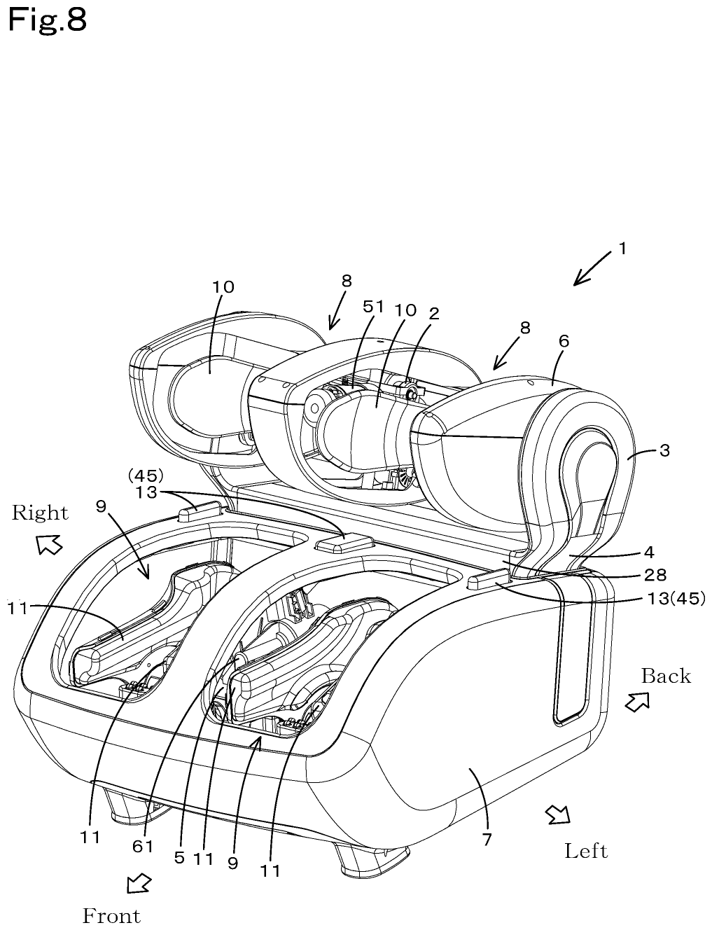

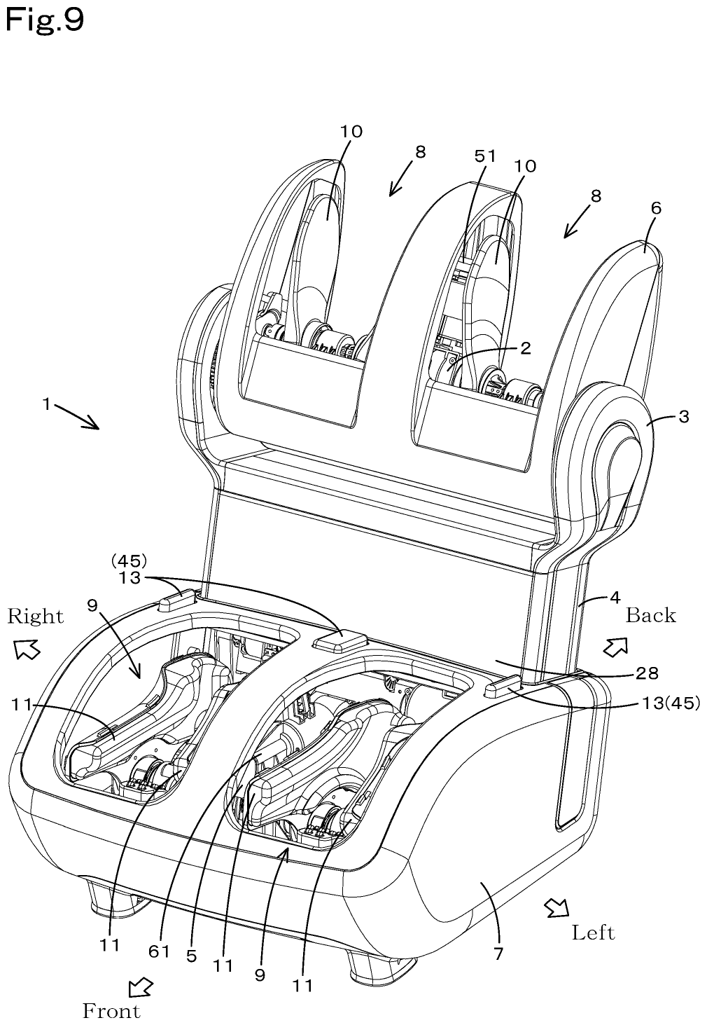

As shown in FIG. 8, the upper casing 6 is an elongated member made to have a forwardly-extending configuration at the “first position”, and have an upwardly-extending configuration at the “sixth position” shown in FIG. 9. The upper casing 6 forms an interior space therein to accommodate the upper massaging system 2.

The upper casing 6 is a forked member having its left end, mid-portion in the horizontal direction, and right end extended forward in furcation form. The upper casing 6 is provided at each of its left end, mid-portion in the horizontal direction, and right end with the first massaging member 10 of the upper massaging system 2. That is, the first massaging members 10 are arranged in pairs, with the first massaging members 10 of each pair spaced apart in the horizontal direction.

For cases where the user manually lifts the upper casing 6, the left and right ends of the upper casing 6 each serve as a grip portion which is grasped by user's hand. The grip portion helps the user change the upper massaging position.

The upper casing 6 is supported for rocking motion relative to the lower casing 7 in the front-rear direction by the rockably supporting system 3 (which will hereafter be described in detail). That is, the upper casing 6 is rockable in sync with the axial rocking motion of the horizontal-pointing rock shaft 12 of the built-in upper massaging system 2.

The upper casing 6 is preferably provided at its upper surface (the upper surface of the horizontal mid-portion is desirable) with a switch to permit selection between an actuation mode and a stationary mode for the operation of the leg massager 1. In this case, even when the upper casing 6 is raised as shown in FIG. 9, the switch disposed on the upper surface of the upper casing 6 is easy to look at for the user, ensuring increased visibility of the switch.

In the upper casing 6, a space between the left end and the horizontal mid-portion, as well as a space between the horizontal mid-portion and the right end, serve as the first massaging recess 8. That is, the upper casing 6 is provided with a pair of horizontally-spaced-apart first massaging recesses 8. The first massaging recesses 8 receive the right and left calves C or the right and left thighs T, for example.

For example, at the “first position” shown in FIG. 8, the first massaging recess 8 has an open top, an open front, and an open bottom. Moreover, at the “sixth position” shown in FIG. 9, the first massaging recess 8 has an open top, an open front, and an open rear. Although not shown in the drawings, the paired right-hand and left-hand first massaging recesses 8 are each fitted internally with a lining material which is elastic, flexible, and adequately breathable.

As shown in the drawings e.g. FIGS. 8 to 11, in the leg massager 1 pursuant to the present invention, the upper massaging system 2 can be supported for rocking motion about a horizontal-pointing axis by the rockably supporting system 3. The rockably supporting system 3 will now be described in detail.

As shown in the drawings e.g. FIGS. 8 to 11, the rockably supporting system 3, which is situated above the vertically moving system 4, supports the base-end side of the upper casing 6 (both ends of the upper massaging system 2 in the horizontal direction) for rocking motion about the horizontal-pointing rock shaft 12, so that the front-end side of the elongated upper casing 6 accommodating the upper massaging system 2 can be raised rearward.

The rockably supporting system 3 enables the upper massaging system 2 to rock about the axis of the horizontal-pointing rock shaft 12 in the front-rear direction. The rockably supporting system 3 is housed in the upper casing 6.

The rockably supporting system 3 comprises: a rockably supporting member 15 that supports the upper massaging system 2 for rocking motion in the front-rear direction; the rock shaft 12 mounted with its axis pointing in the horizontal direction; a drive shaft 16 mounted with its axis pointing in the horizontal direction, which is disposed in a location other than the location of the rock shaft 12; a rock drive section 17 for rotatably driving the drive shaft 16; and a rock conversion section 18 for converting a rotational driving force exerted by the drive shaft 16 into rocking motion of the upper massaging system 2.

The rockably supporting member 15 is a substantially U-shaped member having its left and right ends extended upward. The rockably supporting member 15 comprises: a left-hand protruding portion 19 formed so as to extend upward from the left end; and a right-hand protruding portion 20 formed so as to extend upward from the right end.

The rockably supporting member 15 supports, at its left-hand protruding portion 19 and right-hand protruding portion 20, the upper massaging system 2 mounted in suspended fashion for rocking motion in the front-rear direction. The left-hand protruding portion 19 and the right-hand protruding portion 20 are each formed with a hole 21 having a horizontal-pointing axis. The hole 21 rockably supports each end of the rock shaft 12.

Moreover, the left-hand protruding portion 19 and the right-hand protruding portion 20 each have a circular plate-shaped front end. The outer periphery of each of the left-hand protruding portion 19 and the right-hand protruding portion 20 is provided with a gear-wheel member 25 constituting the rock conversion section 18. The gear-wheel member 25 has a meshing engagement with a pinion gear 24 constituting the rock conversion section 18. The pinion gear 24 travels around the gear-wheel member 25.

The rock shaft 12 is mounted with its axis pointing in the horizontal direction, and attached to the upper massaging system 2. The rock shaft 12 is disposed in each of the left-hand protruding portion 19 and the right-hand protruding portion 20. That is, there is provided a pair of left-hand and right-hand rock shafts 12. More specifically, the left-hand rock shaft 12 is inserted into the hole 21 of the left-hand protruding portion 19, and the right-hand rock shaft 12 is inserted into the right-hand protruding portion 20. The rock shafts 12 permit rocking motion of the upper massaging system 2 in the front-rear direction.

The drive shaft 16 is mounted with its axis pointing in the horizontal direction, and placed so as to run between the left-hand protruding portion 19 and the right-hand protruding portion 20 of the rockably supporting member 15. The drive shaft 16 is mounted with its lengthwise intermediate area passing through a gear case 23 of the rock drive section 17. The drive shaft 16 is rotated under a rotational driving force exerted by the rock drive section 17.

The drive shaft 16 is supported at its ends in the horizontal direction by a base body 54 constituting the upper massaging system 2. Moreover, the drive shaft 16 is provided at each of its horizontal ends with a pinion gear 24 constituting the rock conversion section 18.

The rock drive section 17 is attached to a lengthwise intermediate area of the base body 54 constituting the upper massaging system 2. The rock drive section 17 comprises: a rock motor 22 for producing output of a rotational driving force; and a gear case 23 that, while effecting deceleration to a predetermined rpm, transmits the rotational driving force to the drive shaft 16.

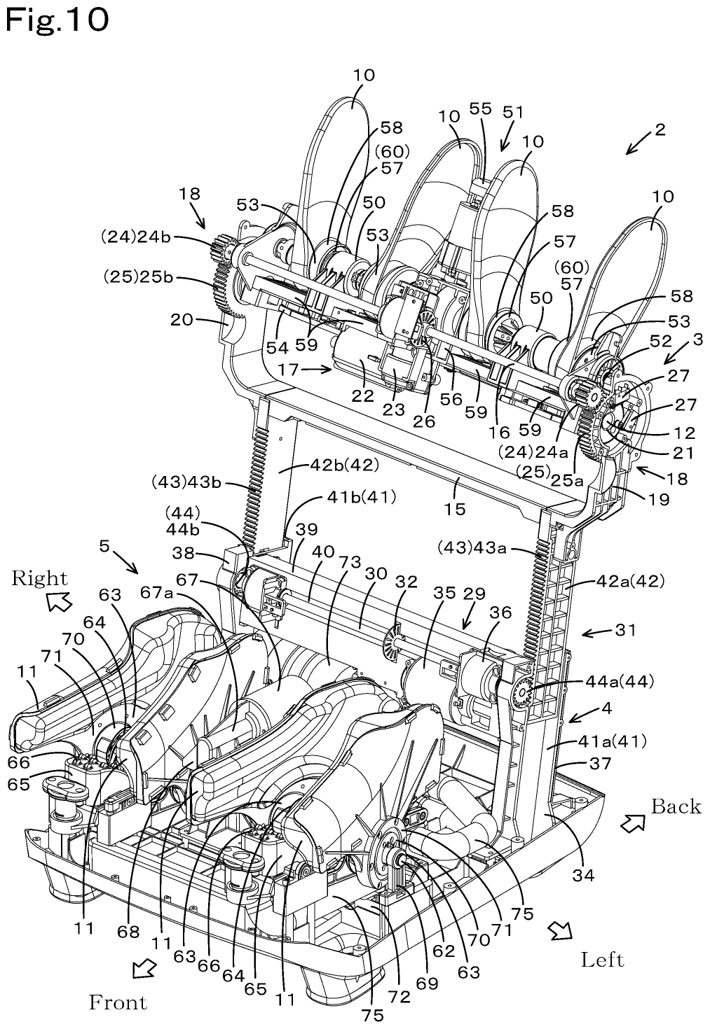

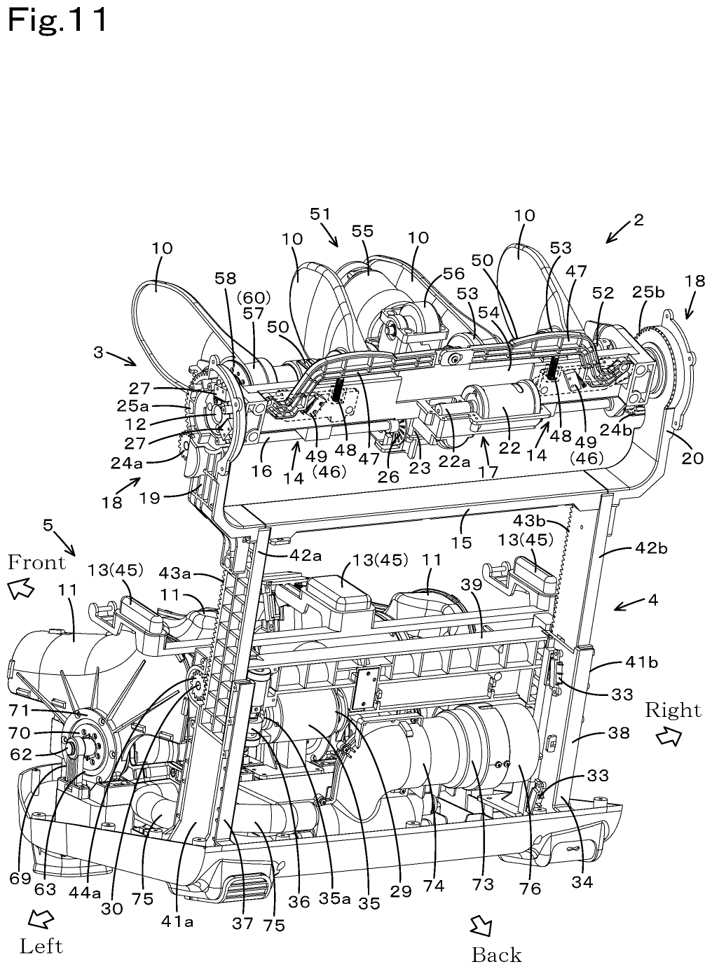

At the “sixth position” (refer to FIG. 10), the rock motor 22 is situated on a wall surface, now being lower wall surface, of the base body 54, with its output shaft 22a pointing leftward. At the “first position” (refer to FIG. 11), the rock motor 22 is situated on a wall surface, now being rear wall surface, of the base body 54.

The gear case 23 receives the drive shaft 16 therethrough, and also accommodates a plurality of gears. Moreover, the output shaft 22a of the rock motor 22 is inserted into the gear case 23. At the “sixth position” (refer to FIG. 10), the gear case 23 is located above the rock motor 22. At the “first position”, the gear case 23 is located forward of the rock motor 22. That is, the rock drive section 17 is located centrally of the base body 54.

The rock conversion section 18 comprises: the pinion gear 24 (planet gear) attached to the drive shaft 16; and the gear-wheel member 25 (sun gear) formed on the outer periphery of each of the left-hand protruding portion 19 and the right-hand protruding portion 20 of the rockably supporting member 15. The rock conversion section 18 has a planetary gear train-like configuration.

In this embodiment, there is provided a gear-wheel member 25a formed on the outer periphery of the front side of the left-hand protruding portion 19 of the rockably supporting member 15. That is, the gear-wheel member 25a may be considered to be secured to the left-hand protruding portion 19. The gear-wheel member 25a has a meshing engagement with a pinion gear 24a attached to the left end of the drive shaft 16.

There is also provided a gear-wheel member 25b formed on the outer periphery of the front side of the right-hand protruding portion 20 of the rockably supporting member 15. That is, the gear-wheel member 25b may be considered to be secured to the right-hand protruding portion 20. The gear-wheel member 25b has a meshing engagement with a pinion gear 24b attached to the right end of the drive shaft 16.

The pinion gear 24 is caused to travel around the gear-wheel member 25 (sun gear) via the drive shaft 16 rotating under a rotational driving force. The movement of the pinion gear 24 permits rocking motion of the upper massaging system 2 in the front-rear direction.

The rockably supporting system 3 further comprises: a shaft-angle detection section 26 for detecting the angle of rotation of the drive shaft 16; a position detection section 27 for detecting the rock limit position of the first massaging member 10; and a stop section that stops the rocking motion of the first massaging member 10 in accordance with the results of detection by the shaft-angle detection section 26 and the position detection section 27.

For example, the stop section may be housed in a control section for controlling the leg massager 1.

The shaft-angle detection section 26 is disposed at a lengthwise intermediate area of the drive shaft 16, and attached to the gear case 23 of the rock drive section 17. The shaft-angle detection section 26 detects a predetermined position of the first massaging member 10 in the front-rear direction. During the rocking motion of the first massaging member 10 in the front-rear direction, the shaft-angle detection section 26 detects the position of the first massaging member 10 upon the angle of rotation of the drive shaft 16 reaching a predetermined level.

Following the completion of position detection by the shaft-angle detection section 26, in accordance with the detection result, the stop section discontinues the rotation of the drive shaft 16 to stop the rocking motion of the first massaging member 10. For example, a rotary encoder is desirable for use as the shaft-angle detection section 26.

The position detection section 27 is attached to each of the left-hand protruding portion 19 and the right-hand protruding portion 20 of the rockably supporting member. The position detection section 27 detects an upper limit position reachable by the first massaging member 10 in rising movement and a lower limit position reachable by the first massaging member 10 in forward-leaning movement.

That is, the position detection section 27 detects the upper or lower limit position of the first massaging member 10 when the pinion gear 24a attached to the left end of the drive shaft 16, now traveling around over the gear-wheel member 25a formed on the left-hand protruding portion 19, reaches the uppermost or lowermost end of the gear-wheel member 25a, as well as when the pinion gear 24b attached to the right end of the drive shaft 16, now traveling around over the gear-wheel member 25b formed on the right-hand protruding portion 20, reaches the uppermost or lowermost end of the gear-wheel member 25b.

Upon the position detection section 27 detecting the upper or lower limit position reachable by the first massaging member 10, in accordance with the detection result, the stop section discontinues the rotation of the drive shaft 16 to stop the rocking motion of the first massaging member 10. For example, a limit switch is desirable for use as the position detection section 27.

The rockably supporting system 3 thus far detailed takes the following transmission path.

That is, on inputting of a rotational driving force outputted from the rock motor 22 to the gear case 23, the rotational driving force is transmitted to the drive shaft 16 within the gear case 23. As the drive shaft 16 is rotated under the transmitted rotational driving force, the pinion gears 24 attached to the left and right ends, respectively, of the drive shaft 16 are rotated correspondingly.

The pinion gear 24a travels around the gear-wheel member 25a formed on the outer periphery of the left-hand protruding portion 19 of the rockably supporting member 15. On the other hand, the pinion gear 24b travels around the gear-wheel member 25b formed on the outer periphery of the right-hand protruding portion 20 of the rockably supporting member 15.

The travel of the pinion gear 24 over the gear-wheel member 25 causes the base body 54 supporting the drive shaft 16 to rock. That is, the pinion gear 24 effects, in conjunction with the gear-wheel member 25, conversion of the rotational driving force into rocking motion. The rocking motion of the base body 54 causes the first massaging member 10 (upper massaging system 2) to rock about the axis of the rock shaft 12 in the front-rear direction.

Thus constructed, the rockably supporting system 3 supports the upper massaging system 2 for rocking motion.

For example, in response to the downward travel of the pinion gear 24 over the gear-wheel member 25, the first massaging member 10 inside the upper casing 6 rocks so as to lean forward. On the other hand, in response to the upward travel of the pinion gear 24 over the gear-wheel member 25, the first massaging member 10 inside the upper casing 6 rocks so as to rise rearward.

Upon the pinion gear 24 reaching the lowermost end of the gear-wheel member 25, the position detection section 27 detects the lower limit position of the first massaging member. In accordance with the result of detection by the position detection section 27, the stop section stops the downward rocking motion of the first massaging member 10. In consequence, the first massaging member 10 inside the upper casing 6 leans forward, and is thus adjusted into the “first position” as shown in FIG. 2, etc. When the first massaging member 10 inside the upper casing 6 remains upward in a position to which it was moved by the vertically moving system 4, the first massaging member 10 is adjusted into the “fourth position” as shown in FIG. 5, etc.

Upon the pinion gear 24 approaching the central part of the gear-wheel member 25 in the vertical direction, the shaft-angle detection section 26 detects that the predetermined position has been reached. In accordance with the result of detection by the position detection section 27, the stop section stops the rocking motion of the first massaging member 10. In consequence, the first massaging member 10 inside the upper casing 6 is directed forward and obliquely upward, and is thus adjusted into the “second position” as shown in FIG. 3, etc. When the first massaging member 10 inside the upper casing 6 remains upward in a position to which it was moved by the vertically moving system 4, the first massaging member 10 is adjusted into the “fifth position” as shown in FIG. 6, etc.

Upon the pinion gear 24 reaching the uppermost end of the gear-wheel member 25, the position detection section 27 detects the upper limit position of the first massaging member. In accordance with the result of detection by the position detection section 27, the stop section stops the upward rocking motion of the first massaging member 10. In consequence, the first massaging member 10 inside the upper casing 6 is directed rearward and obliquely upward, and is thus adjusted into the “third position” as shown in FIG. 4, etc. When the first massaging member 10 inside the upper casing 6 remains upward in a position to which it was moved by the vertically moving system 4, the first massaging member 10 is adjusted into the “sixth position” as shown in FIG. 7, etc.

That is, in this embodiment, in the interest of adaptability to any area of the leg L1 of a user of relatively small build (having a relatively short leg length), such as the calf C1 (first massage target area) and the thigh T1 (second massage target area), the rockably supporting system 3 permits stepwise selection of the position of the first massaging member 10 from among a plurality of positions, ranging from the “first position” (obliquely downward and forward-facing position) to the “third position” (upward-facing position).

Moreover, in this embodiment, in the interest of adaptability to any area of the leg L2 of a user of relatively large build (having a relatively long leg length), such as the calf C2 (first massage target area) and the thigh T2 (second massage target area), the rockably supporting system 3 permits stepwise selection of the position of the first massaging member 10 from among a plurality of positions, ranging from the “fourth position” (obliquely downward and forward-facing position) to the “sixth position” (upward-facing position).

As shown in FIGS. 8 to 11, below the rockably supporting system 3, there is disposed the vertically moving system 4 for moving the upper casing 6 and the upper massaging system 2 in the vertical direction. The vertically moving system 4 is housed in an up-and-down casing 28. The up-and-down casing 28 is disposed at the rear of the lower casing 7. The vertically moving system 4 is housed so as to be freely moved upward out of and retracted into the up-and-down casing 28.

More specifically, the vertically moving system 4 comprises: an up-and-down drive section 29 for producing a driving force to move the upper massaging system 2, etc. in the vertical direction; an up-and-down shaft 30 mounted with its axis pointing in the horizontal direction; up-and-down means 31, disposed below the rockably supporting member 15, for moving the upper massaging system 2, etc. in the vertical direction; and an up-and-down support member 34 that supports the up-and-down drive section 29.

The up-and-down drive section 29 rotates the up-and-down shaft 30 for output of a driving force for moving the upper massaging system 2, etc. in the vertical direction, and transmits the resulting driving force to the up-and-down means 31. The up-and-down drive section 29 comprises: an up-and-down motor 35 for producing output of a driving force (rotational driving force); and a gear case 36 that, while effecting deceleration to a predetermined rpm, transmits the rotational driving force to the up-and-down shaft 30.

The up-and-down motor 35 is located toward the left end of the up-and-down support member 34, with its output shaft 35a pointing leftward. The gear case 36 is located above the up-and-down motor 35. The gear case 36 receives the up-and-down shaft 30 therethrough, and also accommodates a plurality of gears. Moreover, the output shaft 35a of the up-and-down motor 35 is inserted into the gear case 36.

The up-and-down shaft 30 is mounted with its axis pointing in the horizontal direction, and placed so as to run between the left and right ends of the up-and-down support member 34. The up-and-down shaft 30 is located above the up-and-down motor 35, and placed so as to pass through the gear case 36.

The vertically moving system 4 further comprises: a shaft-angle detection section 32 for detecting the angle of rotation of the up-and-down shaft 30; a position detection section 33 for detecting the upward- or downward-movement limit position of the upper massaging system 2; and a stop section that stops the upward or downward movement of the upper massaging system 2 in accordance with the results of detection by the shaft-angle detection section 32 and the position detection section 33.

The stop section may be identical with the stop section provided in the rockably supporting system 3.

The shaft-angle detection section 32 is disposed at a lengthwise intermediate area of the up-and-down shaft 30. The shaft-angle detection section 32 detects a predetermined position of the upper massaging system 2 in the vertical direction. During the vertical movement of the upper massaging system 2, the shaft-angle detection section 32 detects the position of the upper massaging system 2 upon the angle of rotation of the up-and-down shaft 30 reaching a predetermined level.

Following the completion of position detection by the shaft-angle detection section 32, in accordance with the detection result, the stop section discontinues the rotation of the up-and-down shaft 30 to stop the vertical movement of the upper massaging system 2. For example, a rotary encoder is desirable for use as the shaft-angle detection section 32.

The position detection section 33 is attached to each of a left-hand columnar member 37 and a right-hand columnar member 38 of the up-and-down support member. The position detection section 33 detects an upper limit position reachable by the upper massaging system 2 in upward movement and a lower limit position reachable by the upper massaging system 2 in downward movement.

That is, the position detection section 33 detects the upper or lower limit position of the upper massaging system 2 when a pinion gear 44 attached to the left end of the up-and-down shaft 30, now traveling over a rack gear 43 formed on the left-hand columnar member 37, reaches the uppermost or lowermost end of the rack gear 43, as well as when a pinion gear 44 attached to the right end of the up-and-down shaft 30, now traveling over a rack gear 43 formed on the right-hand columnar member 38, reaches the uppermost or lowermost end of the rack gear 43.

When the position detection section 33 detects the upper or lower limit position reachable by the upper massaging system 2, in accordance with the detection result, the stop section discontinues the rotation of the up-and-down shaft 30 to stop the upward or downward movement of the upper massaging system 2. For example, a limit switch is desirable for use as the position detection section 33.

The up-and-down support member 34 is a columnar member formed in upstanding condition on the bottom plate of the lower casing 7. The up-and-down support member 34 comprises: the left-hand columnar member 37 formed in upstanding condition on the left-hand area of the bottom plate of the lower casing 7; the right-hand columnar member 38 formed in upstanding condition on the right-hand area of the bottom plate of the lower casing 7; and a beam member 39 placed so as to run between the left-hand columnar member 37 and the right-hand columnar member 38.

The left-hand columnar member 37 and the right-hand columnar member 38 are each provided with a guide rail 41 constituting the up-and-down means 31. The beam member 39 provides connection between the left-hand columnar member 37 and the right-hand columnar member 38. The beam member 39 is fitted with the shaft-angle detection section 32, the up-and-down motor 35, the gear case 36, etc. Moreover, the beam member 39 is formed with a groove 40 having a horizontal-pointing axis. A bearing engages in the groove 40. The up-and-down shaft 30 is rotatably supported via the bearing.

The up-and-down means 31 is located toward each of the inner left and right ends of the up-and-down casing 28.

The up-and-down means 31 comprises: the guide rail 41 in the form of a vertically-elongated continuous member having a concave cross section; a slider 42 which engages the guide rail 41 for vertical movement; the rack gear 43 mounted with its teeth aligned in the vertical direction; and the pinion gear 44 which moves in the vertical direction while rolling over the rack gear 43.

The guide rail 41 is an elongated rodlike member mounted with its axis pointing in the vertical direction. The guide rail 41 is fitted with the vertically movable slider 42. The guide rail 41 comprises: a left-hand guide rail 41a attached to the left-hand columnar member 37 and a right-hand guide rail 41b attached to the right-hand columnar member 38.

The slider 42 is a columnar member downstanding from the lower end of the rockably supporting member 15. The slider 42 is substantially equal in length to the guide rail 41. The slider 42 is guided along the guide rail 41 into vertical movement. The slider 42 comprises: a left-hand slider 42a formed in downstanding condition on the left-hand area of the rockably supporting member 15; and a right-hand slider 42b formed in downstanding condition on the right-hand area of the rockably supporting member 15.

The rack gear 43 is formed on the front face of the slider 42. The rack gear 43 has a vertically-elongated toothed area. That is, like the slider 42, the rack gear 43 is an elongated member formed on the front face of the slider 42 so as to extend from the upper end to the lower end of the slider 42. The rack gear 43 comprises: a rack gear 43a formed on the front face of the left-hand slider 42a; and a rack gear 43b formed on the front face of the right-hand slider 42b.

The pinion gear 44 is disposed on the front side of the slider 42, and has a meshing engagement with the rack gear 43 attached to the slider 42.

There is provided a pinion gear 44a attached to the left end of the up-and-down shaft 30. The pinion gear 44a has a meshing engagement with the rack gear 43a, and is rotatably fitted in a recess formed in the front side of the guide rail 41a.

There is provided a pinion gear 44b attached to the right end of the up-and-down shaft 30. The pinion gear 44b has a meshing engagement with the rack gear 43b, and is rotatably fitted in a recess formed in the front side of the guide rail 41b.

The guide rail 41, the slider 42, the rack gear 43, and the pinion gear 44 are disposed inside the up-and-down casing 28. Although not shown in the drawings, in order to prevent the slider 42, the pinion gear 44, etc. from accidentally becoming detached from the left-hand columnar member 37 as well as the right-hand columnar member 38, the left-hand columnar member 37 and the right-hand columnar member 38 are each fitted with a cover member for externally covering the slider 42, the pinion gear 44, etc.

The vertically moving system 4 thus far detailed takes the following transmission path.

That is, on inputting of a rotational driving force outputted from the up-and-down motor 35 to the gear case 36, the rotational driving force is transmitted to the up-and-down shaft 30 within the gear case 36. As the up-and-down shaft 30 is rotated under the transmitted rotational driving force, the pinion gear 44a attached to the left end of the up-and-down shaft 30 and the pinion gear 44b attached to the right end of the up-and-down shaft 30 are rotated correspondingly.

The pinion gear 44a travels over the rack gear 43a formed on the front face of the left-hand slider 42a. The pinion gear 44b travels over the rack gear 43b formed on the front face of the right-hand slider 42b.

The travel of the pinion gear 44a over the rack gear 43a causes the left-hand slider 42a to move upward or downward. The travel of the pinion gear 44b over the rack gear 43b causes the right-hand slider 42b to move upward or downward. That is, the pinion gear 44 effects, in conjunction with the gear-wheel member 25, conversion of the rotational driving force into vertical movement. The upward or downward movement of the left-hand slider 42a and the right-hand slider 42b causes the upper massaging system 2 to move in the vertical direction.

Thus constructed, the vertically moving system 4 supports the upper massaging system 2 for vertical movement.

For example, in response to the downward travel of the pinion gear 44 over the rack gear 43, the upper massaging system 2 inside the upper casing 6 moves downward. On the other hand, in response to the upward travel of the pinion gear 44 over the rack gear 43, the upper massaging system 2 inside the upper casing 6 moves upward.

Upon the pinion gear 44 reaching the lowermost end of the rack gear 43, the position detection section 33 detects the lower limit position of the upper massaging system. In accordance with the result of detection by the position detection section 33, the stop section stops the downward movement of the upper massaging system 2 (upper casing 6) which is coming near the lower casing 7. The up-and-down casing 28 is retracted into the lower casing 7. Now the massager is ready for operation of switching among the “first to third positions” shown in FIGS. 2 to 4, for example.

Upon the pinion gear 44 reaching a predetermined area of the rack gear 43 in the vertical direction, the shaft-angle detection section 32 detects that the predetermined position has been reached. In accordance with the result of detection by the position detection section 33, the stop section stops the vertical movement of the upper massaging system 2. In consequence, the upper massaging system 2 (upper casing 6) is brought to a stop in the predetermined position.

Upon the pinion gear 44 reaching the uppermost end of the rack gear 43, the shaft-angle detection section 32 detects the upper limit position of the upper massaging system. In accordance with the result of detection by the position detection section 33, the stop section stops the upward movement of the upper massaging system 2 (upper casing 6) which is moving away from the lower casing 7. The up-and-down casing 28 is moved up out of the lower casing 7. Now the massager is ready for operation of switching among the “fourth to sixth positions” shown in FIGS. 5 to 7, for example.

Moreover, the leg massager 1 according to this embodiment includes the safety mechanism 13 that stops the vertically moving system 4 in case of trouble during the downward movement of the upper casing 6 (in unusual cases, as for example where a load that hinders the downward movement of the upper casing 6 is applied).

The safety mechanism 13 comprises: a lower detection section 45 for detecting a hindrance to the downward movement of the upper casing 6; and a stop section that stops the vertically moving system 4 in accordance with the result of detection by the lower detection section 45.

The lower detection section 45 detects an external load exceeding a predetermined level, such as the presence of a foreign object between the downwardly-moving upper casing 6 and the lower casing 7 (upwardly-pressing load). Moreover, the lower detection section 45 may include a structure for detecting the distance between the downwardly-moving upper casing 6 and the lower casing 7. The lower detection section 45 is disposed on the upper part of the lower casing 7 (lower massaging system 5). The lower detection section 45 is a pushbutton-like member protruding upward from the lower casing 7. The lower detection section 45 is located below that part of the upper casing 6 which protrudes forward. For example, a limit switch is desirable for use as the lower detection section 45.

The stop section stops the vertically moving system 4 when the lower detection section 45 detects the presence of a foreign object between the downwardly-moving upper casing 6 and the lower casing 7. The stop section may be identical with the stop section provided in the rockably supporting system 3.

That is, in the operation of moving the upper casing 6 downward, when the lower detection section 45 detects a downward load, the safety mechanism 13 determines that a foreign object (for example, a hand of a person other than the user (a child, for instance)) gets caught in the gap between the upper casing 6 and the lower casing 7, and immediately deactivate the vertically moving system 4 to stop the downward movement of the upper casing 6 and the upper massaging system 2.

Thus, the safety mechanism 13 prevents human hand, etc. from becoming lodged between the casings, ensuring avoidance of application of a load to the upper casing 6 and the upper massaging system 2.

Moreover, the leg massager 1 according to this embodiment includes the stop mechanism 14 that stops the vertically moving system 4 upon the upper casing 6 reaching a predetermined position during its upward movement.

The stop mechanism 14 comprises: an upper detection section 46 for detecting the arrival of the upwardly-moving upper casing 6 at a predetermined position; and a stop section that stops the vertically moving system 4 in accordance with the result of detection by the upper detection section 46.

For example, the upper detection section 46 detects the arrival of the upper casing 6 (upper massaging system 2) at a predetermined position.

Upon the upper detection section 46 detecting the arrival of the upper casing 6 at the predetermined position, the stop section stops the vertically moving system 4. The stop section may be identical with the stop section provided in the safety mechanism 13.

Moreover, the stop mechanism 14 is configured to stop the vertically moving system 4 in case of trouble during the upward movement of the upper casing 6 (in unusual cases, as for example where a load that hinders the upward movement of the upper casing 6 is applied). The stop mechanism 14 comprises: a plate member 47 which moves downward on contact with a foreign object; and a resilient member 48 (spring) which is brought into compression by the plate member 47. The resilient member 48 (spring) relieves a sudden shock to protect a limit switch 49 from damage.

That is, in the operation of moving the upper casing 6 upward, for example, when there arises an external load exceeding a predetermined level, such as the contact of the plate member 47 with user's leg L (thigh T, for instance) (downwardly-pressing load), the plate member 47 moves downward to bring the spring 48 into compression, and also makes contact with the limit switch 49. Upon detection of the downward load via the limit switch 49, the stop mechanism 14 determines that the load hinders the upward movement of the upper casing 6, and immediately deactivates the vertically moving system 4 to stop the upward movement of the upper casing 6 and the upper massaging system 2. The stop mechanism 14 avoids application of a load to the upper casing 6 and the upper massaging system 2.

The upper casing 6 accommodates the upper massaging system 2. Various mechanisms may be adopted for use as the upper massaging system 2.

As shown in FIGS. 10 and 11, in this embodiment, the upper massaging system 2 comprises: a pair of right-hand and left-hand first massaging members 10 by which an upper massage target area (leg L) such as calf C and thigh T is held at its sides; a pressing member 50 for performing massage from behind the calf C or thigh T; a first drive section 51 for producing a rotational driving force; a first rotation shaft 52 which is rotated under the rotational driving force from the first drive section 51; and a first conversion section 53 for converting a rotational driving force from the first rotation shaft 52 into rocking motion of the first massaging members 10.

The upper massaging system 2 is attached to the base body 54. The base body 54 is attached to the supporting member of the rockably supporting system 3. That is, the upper massaging system 2 is disposed in suspended fashion above the vertically moving system 4, and supported, via the base body 54, for rocking motion 7 in the front-rear direction by the supporting member of the rockably supporting system 3.

Each first massaging member 10 is constructed of an elongated platy material. The first massaging members 10 are arranged in pairs, with the first massaging members 10 of each pair spaced apart in the horizontal direction to receive a massage target area such as the calf C or the thigh T in between. The paired first massaging members 10 perform squeezing (pressing) massage on the calf C or the thigh T.

At the “first position” shown in FIG. 2, etc. or the “fourth position” shown in FIG. 5, etc., the first massaging members 10 have their front ends directed forward for massaging the calf C1, C2 (first massage target area), etc.

At the “second position” shown in FIG. 3, etc. or the “fifth position” shown in FIG. 6, etc., the first massaging members 10 have their front ends directed obliquely upward and forward for massaging the knee-side part of the thigh T1, T2 (second massage target area), etc.

Moreover, at the “third position” shown in FIG. 4, etc. or the “sixth position” shown in FIG. 7, etc., the first massaging members 10 have their front ends directed obliquely upward and rearward for massaging the joint-side part of the thigh T1, T2 (second massage target area), etc.

The pressing member 50 is interposed between the paired right-hand and left-hand first massaging members 10, and rotatably attached to the first rotation shaft 52. The outer periphery of the pressing member 50 is provided with a projection directed outward in a radial direction.

A set of a pair of the first massaging members 10 and the pressing member 50 is assigned to each of the right-hand and left-hand first massaging recesses 8 of the upper casing 6; that is, a total of two such sets are provided. The first massaging member 10 pair and the pressing member 50 are unitarily moved up and down by the vertically moving system 4, ensuring efficacious pressing massage on any of massage target areas such as the calf C and the thigh T.

The first drive section 51 rotates the first rotation shaft 52 to drive the first massaging member 10 and the pressing member 50. The first drive section 51 comprises: a drive motor 55 for producing output of a driving force (rotational driving force); and a gear case 56 that, while effecting deceleration to a predetermined rpm, transmits the rotational driving force to the first rotation shaft 52.

The drive motor 55 is located between the right-hand first massaging member 10 pair and the left-hand first massaging member 10 pair, or equivalently located centrally of the upper massaging system 2. At the “sixth position” shown in FIG. 10, the drive motor 55 stays with its output shaft pointing downward, and the gear case 56 is located below the drive motor 55. The gear case 56 receives the first rotation shaft 52 therethrough, and also accommodates a plurality of gears. Moreover, the output shaft of the drive motor 55 is inserted into the gear case 56.

In a location other than the location of the drive shaft 16, the first rotation shaft 52 is mounted with its axis pointing in the horizontal direction, and placed so as to run between the left-hand protruding portion 19 and the right-hand protruding portion 20 of the rockably supporting member 15. The first rotation shaft 52 is mounted with its lengthwise intermediate area passing through the gear case 56 of the drive section. The first rotation shaft 52 is rotated under a rotational driving force from the first drive section 51.

The first conversion section 53 comprises: a first boss portion 57 that rotates unitarily with the first rotation shaft 52; a first annular fit portion 58, located toward the base end of the first massaging member 10, fitted externally to the first boss portion 57 for relative rotation; and a first restraint portion 59 for restraining the first massaging member 10 from rotating concurrently with the rotation of the first rotation shaft 52.

The first boss portion 57 is provided at its outer periphery with a cam face. In the design of the cam face, a face inclined with respect to the first rotation shaft 52 may be formed, so that the cam face undergoes inclined rotation relative to the axis of the first rotation shaft 52. The first annular fit portion 58 has an inside diameter which is substantially equal to or slightly greater than the outside diameter of the first boss portion 57. The rotation (inclined rotation) of the first boss portion 57 externally fitted with the first annular fit portion 58 imparts rocking motion to the first massaging member 10.

The first restraint portion 59 is located toward the base end of the first annular fit portion 58. The first restraint portion 59 comprises: a first restraint pin attached to the first annular fit portion 58; and a first restraint slot, formed in the base body 54, for receiving the first restraint pin for sliding motion in the horizontal direction. With the side-to-side rocking motion of the first restraint pin within the first restraint slot in substantially the horizontal direction, the first annular fit portion 58 is restrained from rotating concurrently with the rotation of the first boss portion 57.

Moreover, the upper massaging system 2 of this embodiment includes a gap-width adjustment mechanism 60 for adjustment of the width of the gap between the paired first massaging members 10.

The gap-width adjustment mechanism 60 is located toward the base end of the first massaging member 10, and located above the first rotation shaft 52. Although not shown in the drawings, the gap-width adjustment mechanism 60 preferably comprises: a threaded screw portion having external teeth in helical form, which is attached to the first rotation shaft 52; and an internally toothed member located toward the first boss portion 57, which threadedly engages the threaded screw portion.

The horizontal movement of the internally toothed member-equipped first boss portion 57 over the threaded screw portion enables the first massaging member 10 to move in the horizontal direction, thus permitting free adjustment of the width of the gap between the paired first massaging members 10.

The placement of the gap-width adjustment mechanism 60 provides adaptability to the legs L of varying shapes, such as calves C and thighs T of varying sizes and thicknesses.

Although another mechanism may be adopted for use as the gap-width adjustment mechanism 60, the mechanism just described, i.e. the mechanism disclosed in Japanese Unexamined Patent Publication JP-A 2017-153734 is most desirable for use. In the design of the gap-width adjustment mechanism 60, self-adjusting capability may be incorporated therein. Moreover, with consideration given to variations in the shape (dimensions) of the foot F among users, the lower massaging system 5, which will hereafter be described, may also be provided with the gap-width adjustment mechanism 60.

The lower casing 7 has a domical (convex) form with an upwardly-curved upper surface. The lower casing 7 accommodates the lower massaging system 5, etc. On the inner bottom surface of the lower casing 7, there is disposed the up-and-down support member 34 of the vertically moving system 4 in upstanding condition. The lower casing 7 has a pair of horizontally-spaced-apart second massaging recesses 9 opening into the upper surface thereof. The paired right-hand and left-hand second massaging recesses 9 receive user's right and left feet F, respectively. Although not shown in the drawings, the second massaging recesses 9 are each fitted internally with a lining material which is elastic, flexible, and adequately breathable.

The lower casing 7 accommodates the lower massaging system 5. Various mechanisms may be adopted for use as the lower massaging system 5.

As shown in FIGS. 10 and 11, in this embodiment, the lower massaging system 5 comprises: a pair of right-hand and left-hand second massaging members 11 by which a massage target area including the foot F1 (the toe-to-heel region of the leg L) is held at its sides; a second drive section 61 for producing a rotational driving force; a second rotation shaft 62 which is rotated under the rotational driving force from the second drive section 61; and a second conversion section 63 for converting a rotational driving force from the second rotation shaft 62 into rocking motion of the second massaging members 11.

The lower massaging system 5 further comprises: a sole massaging roller 64 for performing pressing massage on the lengthwise central area of user's sole S (the arch (of the foot) A); and a pair of sole massaging members 65 that are disposed forward of and behind the sole massaging roller 64, respectively. The lower massaging system 5 is attached to the bottom part of the lower casing 7.

The second massaging members 11 are located toward the bottom part of the lower casing 7, and located above the second rotation shaft 62. Each second massaging member 11 is a platy member elongated in the front-rear direction so as to conform to the shape of the foot F, including the toe, the instep, the ankle, and the heel, and curved so as to encase the foot F. The second massaging members 11 are arranged in pairs, with the second massaging members 11 of each pair spaced apart in the horizontal direction to receive a massage target area, i.e. the foot F in between. The paired second massaging members 11 perform squeezing (pressing) massage on the foot F.

The sole massaging roller 64 is interposed between the paired right-hand and left-hand second massaging members 11, and rotatably attached to the second rotation shaft 62. The outer periphery of the sole massaging roller 64 is provided with a projection directed outward in a radial direction. In response to the rotation of the second rotation shaft 62, the sole massaging roller 64 is rotated to apply a pressing force for massage to the arch (of the foot) A.

The paired sole massaging members 65 respectively disposed forward of and behind the sole massaging roller 64 each have a rectangular frame form. The front-side sole massaging member 65 and the rear-side sole massaging member 65 are coupled to each other. Each sole massaging member 65 is attached to the second rotation shaft 62. That part of the sole massaging member 65 which faces the front part, as well as the rear part, of the sole S is provided with a plurality of massaging pieces 66 in projection form. In response to the rotation of the second rotation shaft 62, the sole massaging members 65 are rocked, with the massaging pieces 66 performing pressing massage on the sole S.

The second drive section 61 rotates the second rotation shaft 62 to drive the second massaging member 11 and the sole massaging roller 64. The second drive section 61 comprises: a drive motor 67 for producing output of a driving force (rotational driving force); and a gear case 68 that, while effecting deceleration to a predetermined rpm, transmits the rotational driving force to the second rotation shaft 62.

The drive motor 67 is located between the right-hand second massaging member 11 pair and the left-hand second massaging member 11 pair, or equivalently located centrally of the lower massaging system 5. The drive motor 67 is mounted with its output shaft 67a pointing forward. The gear case 68 is disposed forward of the drive motor 67. The gear case 68 receives the second rotation shaft 62 therethrough, and also accommodates a plurality of gears. Moreover, the output shaft 67a of the drive motor 67 is inserted into the gear case 68.

The second rotation shaft 62 is mounted with its axis pointing in the horizontal direction, and placed so as to run between the left and right ends of the lower casing 7. The second rotation shaft 62 is rotatably supported by a lower support member 69 disposed in upstanding condition on the bottom part of the lower casing 7. The second rotation shaft 62 is mounted with its lengthwise intermediate area passing through the gear case 68 of the drive section. The second rotation shaft 62 is rotated under a rotational driving force from the second drive section 61.

The second conversion section 63 comprises: a second boss portion 70 that rotates unitarily with the second rotation shaft 62; a second annular fit portion 71, located toward the base end of the second massaging member 11, fitted externally to the second boss portion 70 for relative rotation; and a second restraint portion 72 for restraining the second massaging member 11 from rotating concurrently with the rotation of the second rotation shaft 62.

The second boss portion 70 is provided at its outer periphery with a cam face. In the design of the cam face, a face inclined with respect to the second rotation shaft 62 may be formed, so that the cam face undergoes inclined rotation relative to the axis of the second rotation shaft 62. The second annular fit portion 71 has an inside diameter which is substantially equal to or slightly greater than the outside diameter of the second boss portion 70. The rotation (inclined rotation) of the second boss portion 70 externally fitted with the second annular fit portion 71 imparts rocking motion to the second massaging member 11.

The second restraint portion 72 is located toward the base end of the second annular fit portion 71. The second restraint portion 72 comprises: a second restraint pin attached to the second annular fit portion 71; and a second restraint slot, formed in the bottom part of the lower casing 7, for slidably receiving the second restraint pin. With the side-to-side rocking motion of the second restraint pin within the second restraint slot in substantially the horizontal direction, the second annular fit portion 71 is restrained from rotating concurrently with the rotation of the second boss portion 70.

The leg massager 1 according to this embodiment further includes a heating device 73 for making the interior of the lower casing 7 warm. The heating device 73 comprises: a hot-air heater 74 for warming of air; and a hose 75 for blowing warmed air into the lower casing 7. The hot-air heater 74 comprises: a heat source-generating section for generating a heat source, such as a heating wire (nichrome wire); an air suction fan 76; a drive motor for driving the fan 76; and a safety arrangement (such as a thermostat or thermal fuse). Air warmed by the hot-air heater 74 is blown into the lower casing 7 by the hose 75, and, the air diffused in the interior of the lower casing 7 warms areas around the second massaging member 11, thereby performing thermomassage on a massage target area such as the foot F.

Thus, the leg massager 1 pursuant to the present invention achieves easy changing of massaging positions and is also capable of adapting to user's legs of varying lengths by using a position-adjustable massaging system for performing massage on massage target areas of user's legs.

That is, as seen from the foregoing description of the present invention, the upper massaging system 2 is supported for rocking motion about a horizontal-pointing axis by the rockably supporting system 3, and is also caused to move up and down by the vertically moving system 4. This arrangement permits stepwise selection of the massaging position from among a plurality of positions (the “first to sixth positions”) in the front-rear direction, that is; the first massaging member 10 is capable of changing its position. Thus, the upper massaging system 2 is capable of performing pressing massage on any of a massage target area of the leg L1, such as the calf C1 and the thigh T1, and a massage target area of the leg L2, such as the calf C2 and the thigh T2, ensuring that the leg massager is adaptable to both of the leg L1 of a user of relatively small build (having a relatively short leg length) sitting in a chair, etc. and the leg L2 of a sitting user of relatively large build (having a relatively long leg length), for example.

Moreover, while the first massaging members 10 are being rocked in the front-rear direction in the range from the calf C through the thigh T, a massage target area including the calf C and the thigh T held between the massaging members can be subjected to a squeezing force for pressing massage on a continuous basis. That is, the leg massager achieves efficacious massage treatment for the entire massage target area in the range from the calf C through the thigh T via the paired right-hand and left-hand first massaging members 10 and the pressing member 50.

The leg massager is also capable of performing pressing massage on the foot F via the lower massaging system 5 in parallel with the massaging operation just described.

It should be understood that the embodiments as disclosed herein are each considered in all respects as illustrative only and not restrictive.

In particular, for such matters as not explicitly specified in the disclosure of the embodiment, for example, for working and operating conditions, and the dimensions and weights of structural components, etc., use has been made of credible requirements that do not depart from practical ranges adopted in the light of the common knowledge of those skilled in the art. For example, the lengths of the massaging members 10 and 11 provided in the massaging system 2 and the massaging system 5, respectively, the angles of inclination of the massaging members 10 and 11 relative to the rotation shafts 52 and 64, respectively, the spaced interval between the right-hand and left-hand massaging members 10, as well as the right-hand and left-hand massaging members 11, etc. may be changed on an as needed basis with consideration given to the range of massage target areas.

In the present invention, there is no specific limitation to the basic structural design of the upper massaging system 2 and the lower massaging system 5. That is, various mechanisms may be adopted for use as the upper massaging system 2 and the lower massaging system 5.

Moreover, the leg massager 1 pursuant to the present invention may include an airbag member for performing pressing massage on massage target areas of user's body. For example, there is provided a pair of airbag members, each positioned at corresponding one of the paired first massaging members 10, for performing pressing massage on the calf C, the thigh T, etc., and there is also provided a pair of airbag members, each positioned at corresponding one of the paired second massaging members 11, for performing pressing massage on the foot F.

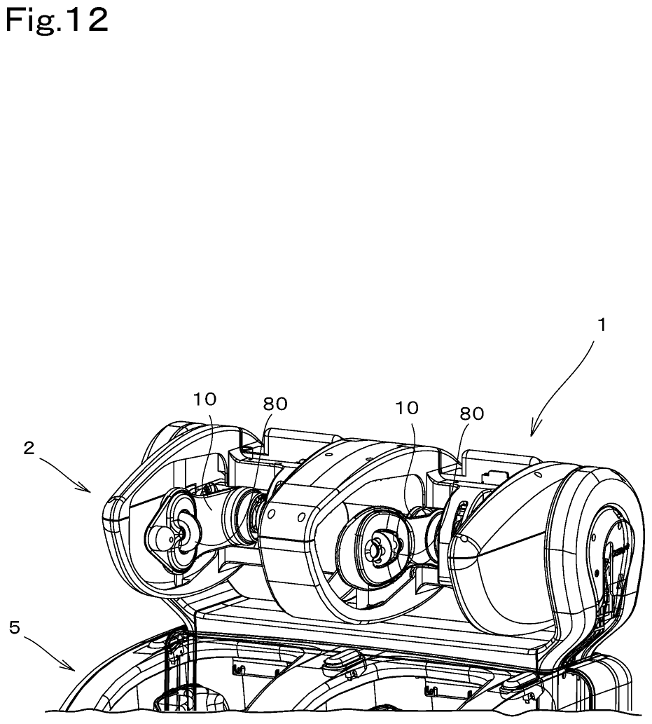

Moreover, as shown in FIG. 12, in the leg massager 1 pursuant to the present invention, each of the paired first massaging members 10 may be provided at the mid-portion of its front end with a rotatable pressing member 80 which is free to rotate in the vertical direction. The rotatable pressing member 80 rotates in response to the vertical movement of the first massaging member 10, thereby performing efficacious pressing massage on a localized region of user's leg (massage target area such as calf, in particular).

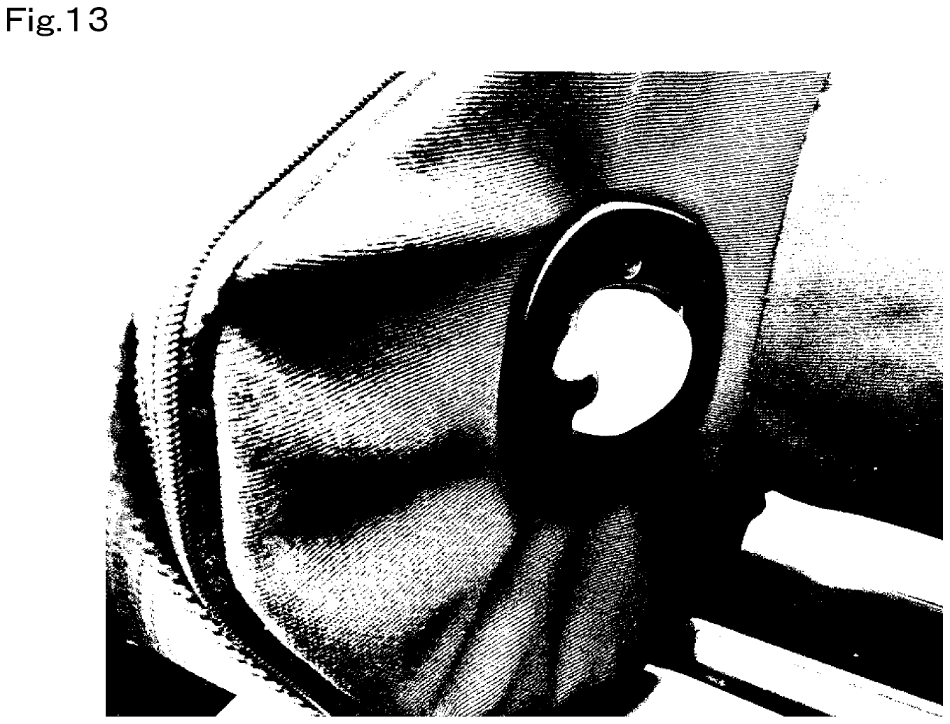

As shown in FIG. 13, in the first massaging member 10 covered with cover members (upper casing 6, cloth, a cushion, etc.), the rotatable pressing member 80 is placed so as to pass through these cover members for direct contact with massage target areas. This makes it possible to perform efficacious pressing massage on a localized region.

Moreover, as described earlier, the upper massaging system 2 of this embodiment includes the gap-width adjustment mechanism 60 for adjustment of the width of the gap between the paired first massaging members 10. Proper control of this gap-width adjustment mechanism 60 permits smooth up-and-down movement of the first massaging member 10. For example, for the passage of the first massaging member 10 through the region corresponding to user's knee, the gap-width adjustment mechanism 60 is actuated so as to widen the gap between the paired right-hand and left-hand first massaging members 10 for smooth passage of the first massaging member 10 through the region corresponding to the knee.

While the foregoing description of the embodiment deals with the case where the upper casing 6 (upper massaging system 2) rocks automatically, the user is able to grasp the upper casing 6 by the front side edge and lift it up. That is, the upper massaging system 2 can be rocked by manual operation. For this case, use can be made of a positioning mechanism capable of stopping the rocking motion of the upper massaging system 2 in the front-rear direction and locking it in a predetermined position. For example, the positioning mechanism may be constructed of a ratchet mechanism capable of step-by-step positional adjustment.

For example, the leg massager 1 pursuant to the present invention may be designed to effect changing and adjustment of the intensity and rhythm of massaging action provided to the leg L. Moreover, the leg massager 1 pursuant to the present invention may be provided with a mechanism for adjusting the rocking angles of the first massaging member 10 and the second massaging member 11.

The upper casing 6 and the lower casing 7 may be made of plastic, and, the first massaging member 10 and the second massaging member 11 may be covered with a fabric or cushion member.

The leg massager 1 pursuant to the present invention may be mounted in massaging equipment in chair form. That is, in a chair massager comprising a seat portion and a backrest portion disposed at the rear of the seat portion, the front side of the seat portion is fitted with the leg massager 1 pursuant to the present invention for vertical rocking motion or for back-and-forth movement.

### REFERENCE SIGNS LIST

1 Leg massager
2 Upper massaging system
3 Rockably supporting system
4 Vertically moving system
5 Lower massaging system
6 Upper casing
7 Lower casing
8 First massaging recess
9 Second massaging recess
10 First massaging member
11 Second massaging member
12 Rock shaft
13 Safety mechanism
14 Stop mechanism
15 Rockably supporting member
16 Drive shaft
17 Rock drive section
18 Rock conversion section
19 Left-hand protruding portion
20 Right-hand protruding portion
21 Hole
22 Rock motor
22a Output shaft
23 Gear case
24 Pinion gear (Planet gear)
24a Pinion gear (Left-hand pinion gear)
24b Pinion gear (Right-hand pinion gear)
25 Gear-wheel member (Sun gear)
25a Gear-wheel member (Left-hand gear)
25b Gear-wheel member (Right-hand gear)
26 Shaft-angle detection section
27 Position detection section
28 Up-and-down casing
29 Up-and-down drive section
30 Up-and-down shaft
31 Up-and-down means
32 Shaft-angle detection section
33 Position detection section
34 Up-and-down support member
35 Up-and-down motor
35a Output shaft
36 Gear case
37 Left-hand columnar member
38 Right-hand columnar member
39 Beam member
40 Groove
41 Guide rail
41a Left-hand guide rail
41b Right-hand guide rail
42 Slider
42a Left-hand slider
42b Right-hand slider
43 Rack gear
43a Rack gear (Left-hand rack gear)
43b Rack gear (Right-hand rack gear)
44 Pinion gear
44a Pinion gear (Left-hand pinion gear)
44b Pinion gear (Right-hand pinion gear)
45 Lower detection section
46 Upper detection section
47 Plate member
48 Resilient member (Spring)
49 Limit switch
50 Pressing member
51 First drive section
52 First rotation shaft
53 First conversion section
54 Base body
55 Drive motor
56 Gear case
57 First boss portion
58 First annular fit portion
59 First restraint portion
60 Gap-width adjustment mechanism
61 Second drive section
62 Second rotation shaft
63 Second conversion section
64 Sole massaging roller
65 Sole massaging member
66 Massaging piece
67 Drive motor
67a Output shaft
68 Gear case
69 Lower support member
70 Second boss portion
71 Second annular fit portion
72 Second restraint portion
73 Heating device
74 Hot-air heater
75 Hose
76 Fan
80 Rotatable pressing member
L Leg
L1 Leg (Short)
L2 Leg (Long)
F Foot
F1 Foot (of short leg)
F2 Foot (of long leg)
C Calf
C1 Calf (of short leg)
C2 Calf (of long leg)
T Thigh
T1 Thigh (of short leg)
T2 Thigh (of long leg)
S Sole
A Arch (of the foot)
R Floor

1. A leg massager, comprising:
a lower massaging system for massaging a massage target area of user's leg including at least user's foot;
an upper massaging system for massaging an upper massage target area located above the massage target area which is massaged by the lower massaging system;
a rockably supporting system that supports the upper massaging system for rocking motion about a horizontal-pointing axis in a front-rear direction; and
a vertically moving system that permits up-and-down movement of the upper massaging system.

2. The leg massager according to claim 1,
wherein the vertically moving system includes a mechanism that brings the upper massaging system to a stop in a predetermined position during the time the vertically moving system is moving the upper massaging system upward.

3. The leg massager according to claim 1,
wherein the rockably supporting system is situated above the vertically moving system, and rockably supports a base-end side of a first massaging member disposed in the upper massaging system so that a front-end side of the first massaging member can be raised rearward via a horizontal-pointing rock shaft.

4. The leg massager according to claim 3,
wherein the first massaging member is folded forward toward a second massaging member disposed in the lower massaging system via the rockably supporting system, at which time the first massaging member is in a position with its front-end side directed forward.

5. The leg massager according to claim 3,
wherein the first massaging member is raised, with its front-end side moved away from a second massaging member disposed in the lower massaging system, via the rockably supporting system, at which time the first massaging member is in an obliquely upward and forward-facing position.

6. The leg massager according to claim 3,
wherein the first massaging member is raised rearward, with its front-end side moved away from a second massaging member disposed in the lower massaging system, via the rockably supporting system, at which time the first massaging member is in an obliquely upward and rearward-facing position.

7. The leg massager according to claim 3,
wherein the first massaging member is folded forward toward a second massaging member disposed in the lower massaging system via the rockably supporting system, and the first massaging member in a forward-leaning position is then moved upward via the vertically moving system, at which time the first massaging member is in a position with its front-end side directed forward.

8. The leg massager according to claim 3,
wherein the first massaging member is moved upward via the vertically moving system, and the first massaging member in an up position is then rocked upward via the rockably supporting system, at which time the first massaging member is in an obliquely upward and forward-facing position.

9. The leg massager according to claim 3,
wherein the first massaging member is moved upward via the vertically moving system, and the first massaging member in an up position is then rocked upward and rearward via the rockably supporting system, at which time the first massaging member is in an obliquely upward and rearward-facing position.

10. The leg massager according to claim 4,
wherein the rockably supporting system is configured to rock the first massaging member in a manner permitting changing of its position from the forward-leaning position to the obliquely upward and forward-facing position, and from there to the obliquely upward and rearward-facing position.

11. The leg massager according to claim 3, further comprising:
a stop mechanism that stops the vertically moving system upon the first massaging member reaching a predetermined position during its upward movement.

12. The leg massager according to claim 11,
wherein the stop mechanism comprises: an upper detection section for detecting an arrival of the upwardly-moving first massaging member at the predetermined position; and a stop section that stops the vertically moving system in accordance with a result of detection by the upper detection section.

13. The leg massager according to claim 3, further comprising:
a safety mechanism that stops the vertically moving system and/or the rockably supporting system in case of trouble during a downward movement of the first massaging member.

14. The leg massager according to claim 13,
wherein the safety mechanism comprises: a lower detection section for detecting a hindrance to the downward movement of the first massaging member; and a stop section that stops the vertically moving system and/or the rockably supporting system in accordance with a result of detection by the lower detection section.

15. The leg massager according to claim 3,
wherein the first massaging member is allowed to move upward, move downward, or rotate in a condition where user's foot is held between the second massaging members.

16. The leg massager according to claim 3,
wherein the first massaging member is shaped in an elongated plate,
wherein the first massaging member is provided at its mid-portion with a rotatable pressing member, which is free to rotate about an axis pointing in a direction longitudinally of the first massaging member, for producing rotational pressure to perform pressing massage on user's leg as a massage target.
